(19) United States
(12) Patent Application Publication
(10) Pub. No.: US 2009/0209510 A1
(43) Pub. Date: Aug. 20, 2009

(54) Novel Method of Treating Hyperlipidemia

(75) Inventors: Tomoyuki Nishimoto, Osaka-shi (JP); Ryuichi Tozawa, Osaka-shi (JP); Takeo Wada, Osaka-shi (JP); Eiichiro Ishikawa, Osaka-shi (JP); Toshiya Nishi, Osaka-shi (JP); Hiroko Yamakawa, Tsukuba-shi (JP)

Correspondence Address: EDWARDS ANGELL PALMER & DODGE LLP, P.O. BOX 55874, BOSTON, MA, 02205, US

(73) Assignee: TAKEDA PHARMACEUTICAL COMPANY LIMITED, OSAKA (JP)

(21) Appl. No.: 11/921,422

(22) PCT Filed: May 31, 2006
(86) PCT No.: PCT/JP2006/311362

§ 371 Date: Feb. 23, 2009

Related U.S. Application Data
(60) Provisional application No. 60/685,871, filed on Jun. 1, 2005.
(60) Provisional application No. 60/728,329, filed on Oct. 20, 2005.

(51) Int. Cl. A61K 31/397; A61K 31/553; C07D 413/02; A61P 3/06

(52) U.S. Cl. 514/210.02; 514/211.05; 540/490

(57) ABSTRACT

A pharmaceutical composition useful for a prevention and/or treatment of hyperlipidemia, which comprises combining an effective amount of squalene synthase inhibitor and HMG-CoA reductase inhibitor is provided.

### TECHNICAL FIELD

The present invention is based on the findings that N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid (hereinafter, abbreviated as Compound X), which is a squalene synthase inhibitor (hereinafter sometimes referred to as an “SSI”) and useful as a preventive and/or therapeutic agent of hyperlipidemia, can potentiate the action of HMG-CoA reductase inhibitor, namely, “statin” (for example, atorvastatin, lovastatin, simvastatin, pravastatin, etc.), which is widely used clinically as a preventive and/or therapeutic agent of hyperlipidemia at present. The invention further relates to a method of treating hyperlipidemia or the like in mammals including animals or humans by use in combination of the squalene synthase inhibitor and the HMG-CoA reductase inhibitor.

### BACKGROUND ART

Hyperlipidemia refers to a state in which the serum lipid concentration elevates abnormally. The serum lipid includes cholesterol, phospholipid, triglyceride (neutral fat) and the like. Specifically, a clinical issue comes out when cholesterol and triglyceride is elevated. Many epidemiological investigations have clearly shown that hypercholesterolemia is one of the three risk factors for atherosclerotic diseases such as myocardial infarction, angina pectoris, cerebral infarction and the like accompanied by hypertension and smoking. Accordingly, proper control of cholesterol level in blood is very important in prevention or treatment of atherosclerotic diseases such as ischemic heart diseases. The above-mentioned HMG-CoA reductase inhibitor has been most widely used clinically hitherto as a medication to lower the blood cholesterol level for prevention and/or treatment of hyperlipidemia.

Current treatment guideline regarding the blood lipid control (NCEP-ATP III, USA, The guideline of Japan Atherosclerosis Society, etc.) suggests that the therapeutic target level for low-density lipoprotein cholesterol (LDL-C) of patients having high risk for ischemic heart disease development to be less than 100 mg/dl. Further, the normal level for triglyceride is less than 150 mg/dl, thus strict lipid control is suggested. However, from the recent results of the large-scale outcome test concerning the active LDL-C lowering therapy, it has been shown that lowering of LDL-C level is effective for lowering a risk of ischemic heart disease development even when LDL-C level is less than 100 mg/dl (PROVE-IT test, TNT test, etc.).

On the other hand, the HMG-CoA reductase inhibitor has clinical risk of side effects based on the fact that it is a medicine which inhibits cholesterol synthesis in vivo by inhibiting the activity of HMG-CoA reductase in the cholesterol biosynthetic pathway and lowering its blood concentration. Specifically, when HMG-CoA reductase is inhibited, not only the biosynthesis of cholesterol but also the biosynthesis of some other components such as ubiquinone, dolichol and heme A, which are necessary for the living body, is also inhibited, so that there are concerns of resulting undesirable side effects (for example, rhabdomyolysis, muscle pain, etc.). Further, side effects such as gastrointestinal disturbance and lowered liver function have been also reported. Therefore, the maximum dosage of the HMG-CoA reductase inhibitor to be administered (for example, atorvastatin and simvastatin: up to 80 mg per day; pravastatin: up to 40 mg per day; pitavastatin: up to 2 mg per day) has been decided based on the dosage for manifesting hepatic toxicity or muscle toxicity and the safety zone in animals and humans. However, since the administration of the HMG-CoA reductase inhibitor at the maximum dosage, which has been approved for administration in humans, may have high frequency of such toxicity, the treatment by high dose of the HMG-CoA reductase inhibitor may not be conducted. Accordingly, in case of administering it for treating hyperlipidemia in practical medication, it is usual that a low dosage is administered to a patient in the beginning and then a higher dosage is administered only when sufficient results are not obtained at the lower dosage. It is general to avoid high dose administration of the HMG-CoA reductase inhibitor as much as possible.

It is expected that the higher dose administration of the HMG-CoA reductase inhibitor will have a potent LDL-C lowering action in order to meet the requirements for the blood lipid control in the current treatment guideline. On the other hand, it is concerned that the high dose therapy of the HMG-CoA reductase inhibitor will increase the risk of manifesting toxicity such as hepatic toxicity, etc. In addition, as for the combination of the HMG-CoA reductase inhibitor and fibrate drug aiming to lower triglyceride, it has been reported that the risk of muscular toxicity such as rhabdomyolysis or the like increased.

Under this circumstance, it is considered that the combination therapy of the HMG-CoA reductase inhibitor with a novel medicine, which makes it possible to treat patients who cannot reach the therapeutic target level of LDL cholesterol with the HMG-CoA reductase inhibitor alone therapy, reduce toxicity risk of the high dose therapy and further improve total lipids including triglyceride, can be an important choice for prevention and/or treatment of hyperlipidemia.

### DISCLOSURE OF THE INVENTION

The present inventors have found unexpectedly in the course of investigating various actions of the Compound X that this compound, when combined with the HMG-CoA reductase inhibitor, potentiates an action of lowering cholesterol and triglyceride as compared with individual administration of the HMG-CoA reductase inhibitor, and reduces hepatic toxicity by the HMG-CoA reductase inhibitor, and completed the present invention.

That is, the invention relates to:

(1) A method for preventing and/or treating hyperlipidemia, which comprises administering to a mammal affected with hyperlipidemia a combination of an effective amount of squalene synthase inhibitor and HMG-CoA reductase inhibitor;
(2) The method according to the above-mentioned (1), wherein the HMG-CoA reductase inhibitor is administered at a high dose in approved dosage;
(3) The method according to the above-mentioned (2), wherein the HMG-CoA reductase inhibitor is administered at a maximum dose in approved dosage;
(4) A method for preventing and/or treating hepatic toxicity caused by administration of HMG-CoA reductase inhibitor, which comprises administering an effective amount of squalene synthase inhibitor to inhibit toxicity caused by the administration of HMG-CoA reductase inhibitor to a mammal administered HMG-CoA reductase inhibitor;
(5) The method according to the above-mentioned (4), wherein the mammal is affected with hyperlipidemia;
(6) The method according to the above-mentioned (1) or (2), wherein the squalene synthase inhibitor is a compound represented by formula:

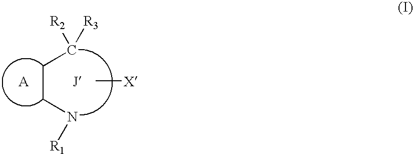

wherein, R₁ is a hydrogen atom or an optionally substituted hydrocarbon group, R₂ and R₃ are the same or different and a hydrogen atom, optionally substituted hydrocarbon group or an optionally substituted heterocyclic group, X′ is a group comprising an optionally esterified carboxyl group, an optionally substituted carbamoyl group, an optionally substituted hydroxy group, an optionally substituted amino group or an optionally substituted heterocyclic residue having a hydrogen atom which can be deprotonated, Ring A is an optionally substituted benzene ring or an optionally substituted heterocyclic ring, Ring J′ is a 7- or 8-membered heterocyclic ring containing 3 or less hetero atoms as ring constituent atoms, and Ring J′ may further have a substituent in addition to R₁, R₂, R₃ and X′;
(7) The method according to the above-mentioned (1) or (2), wherein the squalene synthase inhibitor is N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid;
(8) The method according to the above-mentioned (1) or (2), wherein the HMG-CoA reductase inhibitor is one or more drugs selected from the group consisting of atorvastatin, lovastatin, simvastatin, pravastatin, fluvastatin, rosuvastatin, cerivastatin and pitavastatin;
(9) The method according to the above-mentioned (1) or (2), wherein the mammal affected with hyperlipidemia is HMG-CoA reductase inhibitor intolerant patient;
(10) The method according to the above-mentioned (1) or (2), wherein the mammal affected with hyperlipidemia is a high-risk patient of ischemic heart disease;
(11) The method according to the above-mentioned (1) or (2), wherein the mammal affected with hyperlipidemia is a patient affected with familial hypercholesterolemia;
(12) A pharmaceutical composition for a prevention and/or treatment of hyperlipidemia, which comprises combining an effective amount of squalene synthase inhibitor and HMG-CoA reductase inhibitor;
(13) A pharmaceutical composition for a prevention and/or treatment of hyperlipidemia comprising an effective amount of squalene synthase inhibitor and HMG-CoA reductase inhibitor, which is compounded or packed so as to administer in divided doses, sequentially or simultaneously to a mammal affected with hyperlipidemia;
(14) The pharmaceutical composition according to the above-mentioned (12) or (13), which comprises combining with a high-dose in approved dosage of HMG-CoA reductase inhibitor;
(15) The pharmaceutical composition according to the above-mentioned (12) or (13), which comprises combining with a maximum dose in approved dosage of the HMG-CoA reductase inhibitor;
(16) A method for enhancing an effect on prevention and/or treatment of hyperlipidemia by a HMG-CoA reductase inhibitor, which comprises administering an effective amount of squalene synthase inhibitor to a mammal affected with hyperlipidemia wherein an effective amount of HMG-CoA reductase inhibitor is administered;
(17) Use of squalene synthase inhibitor for the manufacture of a pharmaceutical composition for preventing and/or treating hyperlipidemia which comprises combining an effective amount of squalene synthase inhibitor and HMG-CoA reductase inhibitor;
(18) Use of squalene synthase inhibitor for the manufacture of a pharmaceutical composition for preventing and/or treating hyperlipidemia comprising an effective amount of squalene synthase inhibitor and HMG-CoA reductase inhibitor which is compounded or packed so as to administer in divided doses, sequentially or simultaneously to a mammal affected with hyperlipidemia;
(19) The method according to the above-mentioned (1) or (2), wherein an effective amount of ezetimibe is further administered in combination as third medicament;
(20) The pharmaceutical composition according to the above-mentioned (12) or (13), which comprises further combining an effective amount of ezetimibe as third medicament;
(21) The use of squalene synthase inhibitor for the manufacture of a pharmaceutical composition for preventing and/or treating hyperlipidemia according to the above-mentioned (17) or (18), which comprises further combining an effective amount of ezetimibe as third medicament; and the like.

Compound X is a known compound disclosed, for example, in JP-A No. 9-136880 (Example 36). It has been known that this compound has squalene synthase inhibiting action, and inhibits one step of the same cholesterol biosynthetic pathway as the HMG-CoA reductase inhibitor does (but located downstream of its action point) to suppress cholesterol biosynthesis, which lowers the cholesterol concentration in blood, and thus it is useful for prevention and/or treatment of hyperlipidemia.

JP-A No. 9-136880 discloses that the SSI compounds of the application including Compound X may be used in combination with other various lipid-lowering drugs or cholesterol-lowering drugs in prevention and/or treatment of hyperlipidemia where the use in combination with the HMG-CoA reductase inhibitor is also mentioned. However, no mentions have been made of the active effects of the potentiation of actions and effects by the use in combination of both as compared with the individual administration (pharmacological data are not disclosed, either).

In addition, it has been known that the SSI including Compound X has an action of reducing muscle toxicity by the HMG-CoA reductase inhibitor such as rhabdomyolysis and the like (WO04/064865). Further, it has been known that the SSI including Compound X has an action of increasing ubiquinone, thus it is effective for prevention and/or treatment of organ function disorder and organ insufficiency due to atherosclerotic diseases and cerebro-vascular diseases and the like (WO03/002147). However, it has not been reported that the SSI including Compound X has an organ protective action for drug-induced organ toxicity, particularly liver disorder manifested as side effects of the HMG-CoA reductase inhibitor.

For effects from the use in combination of Compound X and the HMG-CoA reductase inhibitor, the inventors have found that the actions and effects are significantly potentiated in the animal model by combination of both as compared with the individual administration as shown in the serial pharmacological test results below. The effects from the use in combination of Compound X, the SSI and the HMG-CoA reductase inhibitor are said to be unexpected and not assumable from the conventional recognition.

Furthermore, the inventors have also found for the first time that by use in combination of Compound X, the SSI, and the HMG-CoA reductase inhibitor, the hepatic toxicity manifested by the HMG-CoA reductase inhibitor could be suppressed as shown in the pharmacological test results below.

From the findings in the animal model described above, the inventors has reached an invention which is able to achieve medical effects as follows in a human by using the SSI and the HMG-CoA reductase inhibitor in combination for prevention and/or treatment of hyperlipidemia.

1) Improvement of serum lipid can be achieved more strongly, which cannot be achieved by individual administration of the HMG-CoA reductase inhibitor or the SSI, respectively.

2) Side effects of the HMG-CoA reductase inhibitor such as muscle toxicity, hepatic toxicity, or the like can be suppressed. Thereby the HMG-CoA reductase inhibitor in a higher dosage than the conventional dosage can be administered safely to patients.

By such excellent synergic effects, treating with a combination of the SSI and the HMG-CoA reductase inhibitor can control hyperlipidemia more effectively as compared with treating with the HMG-CoA reductase inhibitor alone. That is, by administrating the HMG-CoA reductase inhibitor in combination with the SSI, even at its maximum dosage, lipid lowering action can be potentiated without manifesting hepatic toxicity and muscle toxicity which are concerns for the HMG-CoA reductase inhibitor, thereby providing a novel method of treatment of hyperlipidemia in practical medication. Furthermore, it can be expected that the use in combination with the SSI may open a way to administer the HMG-CoA reductase inhibitor in an amount beyond the maximum dosage approved at present.

It has not been reported so far that a use in combination of the HMG-CoA reductase inhibitor with the SSI can achieve the above-mentioned merits in a human or an animal test. Therefore, the invention provides a novel use of a squalene synthase inhibitor.

### BEST MODE FOR CARRYING OUT THE INVENTION

The preferable examples of each definition in the present invention are as follows.

“Hyperlipidemia” refers to a state in which the serum lipid concentration elevates abnormally. The serum lipid includes cholesterol, phospholipid, triglyceride (neutral fat) and the like. Specifically, a clinical issue comes out when cholesterol and triglyceride is elevated. “Hyperlipidemia” includes hypercholesterolemia, hypertriglyceridemia, and the like.

As mentioned above, a superior prophylactic and/or therapeutic effect of hyperlipidemia can be obtained in the present invention, therefore it is suitable for application to, inter alia, serious diseases in hyperlipidemia. For example, it can advantageously be applied to the cases where the patient (a mammal) affected with hyperlipidemia is a patient having a history of ischemic heart disease, a high-risk patient having plural risk factors of ischemic heart disease such as hypertension, diabetes, obesity and smoking, or a patient affected with familial hypercholesterolemia.

“HMG-CoA reductase inhibitor” in the present invention means, what is called, “statin” such as atorvastatin, lovastatin, simvastatin, pravastatin, fluvastatin, rosuvastatin, pitavastatin, cerivastatin, or the like.

In the present invention, the term of the “high dose in approved dosage” for HMG-CoA reductase inhibitor refers to a dose of higher dosage side including maximum dose, and when there are a number of approved dosages, the term sometimes means plural doses. Generally, the dose falls under a dose exceeding that of HMG-CoA reductase inhibitor which is usually administered to each mammal having a prevention and/or treatment of hyperlipidemia by a single administration of HMG-CoA reductase inhibitor (i.e., usual dose), and it varies from one mammal to another.

The approved dosages of each HMG-CoA reductase inhibitor commercially available at present are as follows.

Atorvastatin: 10, 20, 40, 80 mg/man/day

Simvastatin: 5, 10, 20, 40, 80 mg/man/day

Pravastatin: 10, 20, 40 80 mg/man/day

Fluvastatin: 20, 40, 80 mg/man/day

Lovastatin: 10, 20, 40 mg/man/day

Rosuvastatin: 5, 10, 20, 40 mg/man/day

Pitavastatin: 1, 2 mg/man/day

Examples of the preferable doses of the “high dose in approved dosage” include, but are not limited to, 40, 80 mg/man/day in Atorvastatin, 20, 40, 80 mg/man/day (more preferably 40, 80 mg/man/day) in Simvastatin, 40, 80 mg/man/day in Pravastatin, 40, 80 mg/man/day in Fluvastatin, 20, 40 mg/man/day in Lovastatin, 20, 40 mg/man/day in Rosuvastatin, and 2 mg/man/day in Pitavastatin.

The “HMG-CoA reductase inhibitor intolerant patient” refers to, among patients including both of a “HMG-CoA reductase inhibitor low reactive patient” wherein administration of HMG-CoA reductase inhibitor shows no cholesterol lowering action or insufficient effect and a patient wherein the administration is restricted because of a high incidence rate of side effects, the patients wherein a sufficient prevention and/or treatment of hyperlipidemia cannot be achieved by a usual treatment (for example, the patients who cannot achieve the desired value in Current treatment guideline regarding the blood lipid control (NCEP-ATP III, USA, The guideline of Japan Atherosclerosis Society, etc.)).

As the “squalene synthase inhibitor” to be used in the present invention, any compound can be used as long as it has a squalene synthase inhibitory activity, for example, squalenestatins (e.g., U.S. Pat. Nos. 5,506,262, 5,430,055, 5,409,950, 5369125, JP-A Nos. 7-173166, 9-124655, 9-227566, “Annual Review of Microbiology”, Vol. 49, pp. 607-639, 1995, “Journal of Medicinal Chemistry”, Vol. 38, pp. 3502-3513, 1995, “Journal of Medicinal Chemistry”, Vol. 39, pp. 207-216, 1996, “Journal of Medicinal Chemistry”, Vol. 39, pp. 1413-1422, 1996, etc.), a phosphate compound and a carboxylic acid compound of a substrate analog (e.g., U.S. Pat. Nos. 5,374,628, 5,441,946, 5,428,028, JP-A No. 7-041554, WO95/04025, “Journal of Medicinal Chemistry”, Vol. 38, pp. 2596-2605, 1995, “Arzniemittel-Forschung Drug Research”, Vol. 46, pp. 759-762, 1996, “Journal of Medicinal Chemistry”, Vol. 31, pp. 1869-1871, 1988, “Journal of Medicinal Chemistry”, Vol. 39, pp. 657-660, 1996, “Journal of Medicinal Chemistry”, Vol. 39, pp. 661-664, 1996), carboxylic acid derivatives (e.g., WO97/40006, WO96/33159, WO95/21834, WO97/48701, EP-A Nos. 645377, 645378, 814080, 790235, JP-A Nos. 7-173120, 10-316634, 10-298134, 10-298177, 10-316617, 9-136880, WO2000/00458, WO2001/98282, WO98/29380, “Bioorganic Medicinal Chemistry Letters”, Vol. 5, pp. 1989-1994, 1995, “Bioorganic Medicinal Chemistry Letters”, Vol. 6, pp. 463-466, 1996, “Journal of Medicinal Chemistry”, Vol. 40, pp. 2123-2125, 1997, etc.), an amine-based compound such as quinuclidine derivatives (e.g., U.S. Pat. Nos. 5,385,912, 5,494,918, 5,395,846, 5,451,596, JP-A Nos. 8-134067, 2000-169474, 10-152453, 2000-502716, WO94/03541, WO 94/05660, WO95/35295, WO96/26938, WO95/31458, WO95/00146, WO97/25043, WO98/12170, etc.), and Zaragozic acids, particularly, a compound represented by the formula:

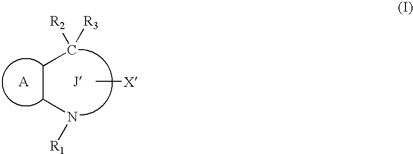

wherein, R₁ is a hydrogen atom or an optionally substituted hydrocarbon group, R₂ and R₃ are the same or different and a hydrogen atom, optionally substituted hydrocarbon group or an optionally substituted heterocyclic group, X′ is a group comprising an optionally esterified carboxyl group, an optionally substituted carbamoyl group, an optionally substituted hydroxy group, an optionally substituted amino group or an optionally substituted heterocyclic residue having a hydrogen atom which can be deprotonated, Ring A is an optionally substituted benzene ring or an optionally substituted heterocyclic ring, Ring J′ is a 7- to 8-membered heterocyclic ring containing 3 or less hetero atoms as ring constituent atoms, and Ring J′ may further have a substituent in addition to R₁, R₂, R₃, and X′; or a compound represented by the formula:

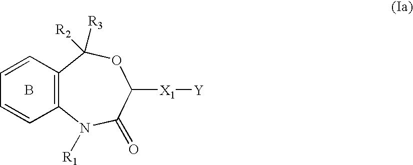

wherein, R₁ is a hydrogen atom or an optionally substituted hydrocarbon group, R₂ and R₃ are the same or different and a hydrogen atom, optionally substituted hydrocarbon group or optionally substituted heterocyclic group, X₁ is a bond or divalent atomic chain, Y is an optionally esterified carboxyl group, an optionally substituted carbamoyl group, an optionally substituted hydroxy group, an optionally substituted amino group or an optionally substituted heterocyclic residue having a hydrogen atom which can be deprotonated, Ring B is an optionally substituted benzene ring; a compound represented by the formula [II]:

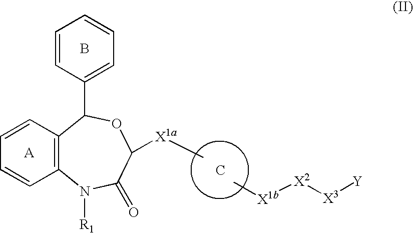

wherein ring A and ring B each represent an optionally substituted benzene ring, ring C represents an optionally further substituted aromatic ring, R¹ represents a lower alkyl group optionally substituted with an optionally substituted hydroxyl group, X^{1a} represents a bond or optionally substituted lower alkylene, X^{1b} represents a bond or optionally substituted lower alkylene, X² represents a bond, —O— or —S—, X³ represents a bond or an optionally substituted divalent hydrocarbon group, and Y represents an optionally esterified or amidated carboxyl group;
or the like is preferably used.

Examples of other squalene synthase inhibitors include A-104109 (Abbott Laboratories),

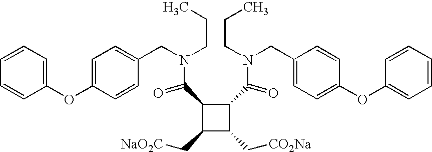

F-10863-A (Zaragozic acid D3, Sankyo Co., Ltd.), bisphosphonic acid derivatives such as ER-28448, ER-27856 (ER-28448 prodrug), and quinuclidine derivatives (Eisai) such as ER-119884 and ER-132781,

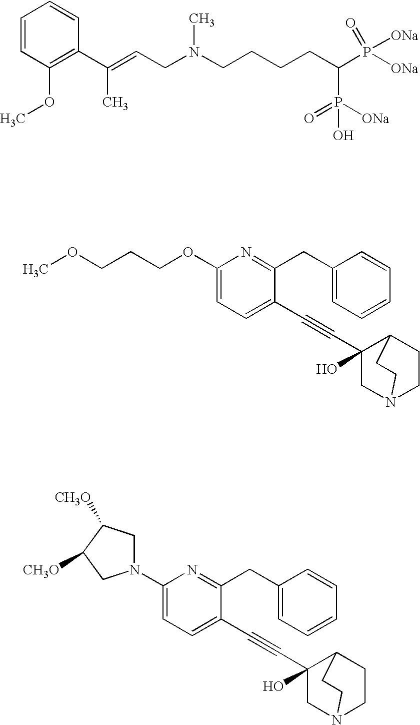

### RPR-107393 and RPR-101821 (Aventis Pharma Ltd.),

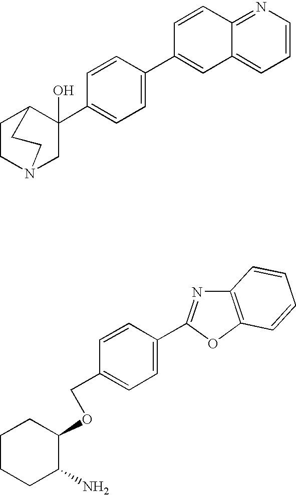

thiadiazole derivatives (NovoNordisk),

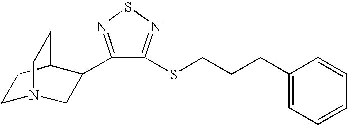

isopropylamine derivatives and quinuclidine derivatives (Yamanouchi Pharmaceutical Co., Ltd.),

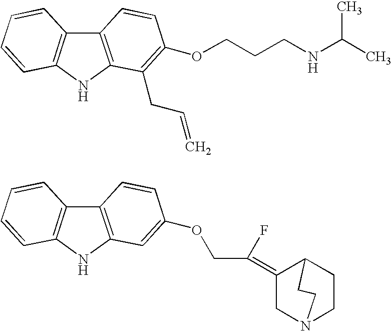

isoquinuclidine derivatives (Kotobuki pharmaceutical Co., Ltd.)

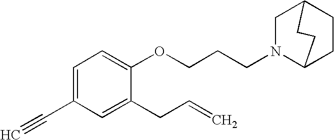

malonic acid derivatives (Nippon Kayaku Co., Ltd.),

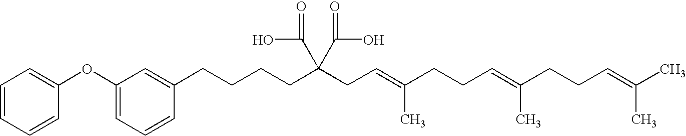

propionyl derivatives (Daiichi Pharmaceutical Co., Ltd.)

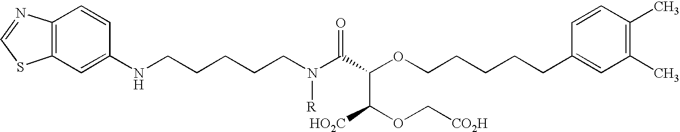

wherein R is hydrogen atom or methyl group,

### SQ-34919, SQ-32709, BMS-187745 and BMS-188494 (Bristol-Myers Squibb Company)

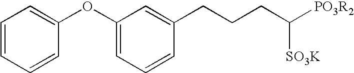

wherein R is potassium atom or —CH₂OCOC(CH₃)₃, J-104118 (Merck & Co., Inc.)

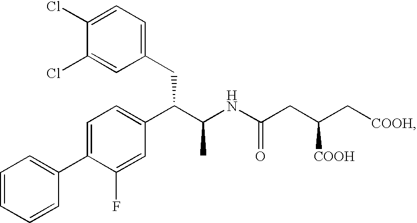

quinuclidine derivatives (AstraZeneca)

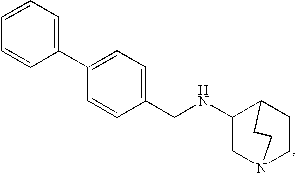

### SDZ-266-806 (Novartis Pharma)

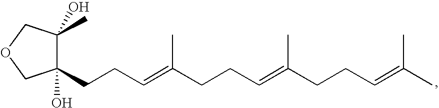

and such squalene synthase inhibitors can be also used in an agent of the present invention.

The “compound having squalene synthase inhibitory activity” used in the present invention can be used in a form of a salt or a prodrug.

As for a “salt” of the compound having squalene synthase inhibitory activity used in the present invention, a pharmaceutically acceptable salt or a physiologically acceptable acid addition salt is preferred. For such salts, for example, inorganic acids (e.g., hydrochloric acid, phosphoric acid, hydrobromic acid, sulfuric acid, etc.) or organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid, etc.) or the like are used. Further, in the case that the “compound having squalene synthase inhibitory activity” used in the present invention has an acidic group such as carboxylic acid or the like, the “compound having squalene synthase inhibitory activity” may form salts with, for example, an inorganic base (e.g., an alkali metal or alkaline earth metal such as sodium, potassium, calcium, magnesium, or ammonia, etc.) or an organic base (e.g., tri-C₁₋₃ alkylamine such as triethylamine, etc.).

The “prodrug” of the compound having squalene synthase inhibitory activity [hereinafter, referred to as “SSI Compound”] used in the present invention or a salt thereof refers to a compound which is converted to the SSI Compound by a reaction in vivo under the physiological condition with an enzyme, a gastric acid or the like, that is, a compound which is converted to the SSI Compound by enzymatic oxidation, reduction, hydrolysis, etc.; a compound which is converted to the SSI Compound by hydrolysis or the like with gastric acid, etc.; or the like. Examples of the prodrug of the SSI Compound include a compound wherein an amino group of the SSI Compound is acylated, alkylated or phosphorylated (e.g., a compound wherein an amino group of the SSI Compound is eicosanoylated, alanylated, pentylaminocarbonylated, (5-methyl-2-oxo-1,3-dioxolen-4-yl)methoxycarbonylated, tetrahydrofuranylated, pyrrolidylmethylated, pivaloyloxymethylated or tert-butylated, etc.); a compound wherein a hydroxy group of the SSI Compound is acylated, alkylated, phosphorylated or borylated (e.g., a compound wherein a hydroxy group of the SSI Compound is acetylated, palmitoylated, propanoylated, pivaloylated, succinylated, fumarylated, alanylated or dimethylaminomethylcarbonylated, etc.); or a compound wherein a carboxyl group of the SSI Compound is esterified or amidated (e.g., a compound wherein a carboxyl group of the SSI Compound is ethylesterified, phenylesterified, carboxymethylesterified, dimethylaminomethylesterified, pivaloyloxymethylesterified, ethoxycarbonyloxyethylesterified, phthalidylesterified, (5-methyl-2-oxo-1,3-dioxolen-4-yl)methylesterified, cyclohexyloxycarbonylethylesterified or methylamidated, etc.); and the like. These compounds can be prepared from the SSI Compound by a per se known method.

In addition, the prodrug of the SSI Compound may be a compound which is converted into the SSI Compound under the physiological conditions as described in “Pharmaceutical Research and Development”, Vol. 7 (Molecular Design), pp. 163-198, published in 1990 by Hirokawa Publishing Co.

Further, the SSI Compound may be hydrated.

When the optically active form of the SSI Compound is needed, it can be obtained, for example, by using an optically active starting material, or by using a conventional method to optically resolve the racemic form of the SSI Compound. Further, when the SSI Compound contains an asymmetric carbon in its molecule and has two stereoisomers of R-configuration and S-configuration, any isomer or a mixture thereof is included within the scope of the present invention.

In the formulae (I) and (Ia), examples of the hydrocarbon group in the “optionally substituted hydrocarbon group” represented by R₁ include an aliphatic chain (acyclic) hydrocarbon group, an alicyclic hydrocarbon group and an aryl group, and among these, aliphatic chain hydrocarbon group is preferred.

The aliphatic chain hydrocarbon group of the hydrocarbon group includes a linear or branched aliphatic hydrocarbon group such as an alkyl group, an alkenyl group, and an alkynyl group. Among these, the branched alkyl group is preferred. Examples of the alkyl group include C₁₋₇ alkyl such as methyl, ethyl, n-propyl, isopropyl, n-butyl, isobutyl, sec-butyl, tert-butyl, n-pentyl, isopentyl, neopentyl, 1-methylpropyl, n-hexyl, isohexyl, 1,1-dimethylbutyl, 2,2-dimethylbutyl, 3,3-dimethylbutyl, 3,3-dimethylpropyl, 2-ethylbutyl, n-heptyl and the like. Inter alia, C₃₋₅ alkyl such as n-propyl, isopropyl, isobutyl, neopentyl and the like is preferred, and isobutyl, neopentyl and the like are particularly preferred. Examples of the alkenyl group include C₂₋₆ alkenyl such as vinyl, allyl, isopropenyl, 2-methylallyl, 1-propenyl, 2-methyl-1-propenyl, 2-methyl-2-propenyl, 1-butenyl, 2-butenyl, 3-butenyl, 2-ethyl-1-butenyl, 2-methyl-2-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl, 5-hexenyl and the like. Inter alia, vinyl, allyl, isopropenyl, 2-methylallyl, 2-methyl-1-propenyl, 2-methyl-2-propenyl, 3-methyl-2-butenyl and the like are particularly preferred. Examples of the alkynyl group include C₂₋₆ alkynyl such as ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like, inter alia ethynyl, 1-propynyl, 2-propynyl and the like are particularly preferred.

The alicyclic hydrocarbon group of the hydrocarbon group includes a saturated or unsaturated alicyclic hydrocarbon group such as a cycloalkyl group, a cycloalkenyl group, a cycloalkadienyl group and the like. As the cycloalkyl group, a C₃₋₉ cycloalkyl group is preferred, and examples thereof include cyclopropyl, cyclobutyl, cyclopentyl, cyclohexyl, cycloheptyl, cyclooctyl, cyclononyl and the like. Among these, a C₃₋₆ cycloalkyl group such as cyclopropyl, cyclobutyl, cyclopentyl and cyclohexyl is preferred. Examples of the cycloalkenyl group include a C₅₋₆ cycloalkenyl group such as 2-cyclopenten-1-yl, 3-cyclopenten-1-yl, 2-cyclohexen-1-yl, 3-cyclohexene-1-yl, 1-cyclobuten-1-yl and 1-cyclopenten-1-yl. Examples of the cycloalkadienyl group include a C₅₋₆ cycloalkadienyl group such as 2,4-cyclopentadien-1-yl, 2,4-cyclohexadien-1-yl and 2,5-cyclohexadien-1-yl.

The aryl group of the hydrocarbon group includes a C₆₋₁₆ monocyclic or fused polycyclic aromatic hydrocarbon group such as phenyl, naphthyl, anthryl, phenanthryl and acenaphthylenyl, inter alia, a C₆₋₁₀ aryl group such as phenyl, 1-naphthyl and 2-naphthyl is particularly preferred.

The substituent of the “optionally substituted hydrocarbon group” represented by R₁ includes an optionally substituted aryl group, an optionally substituted cycloalkyl group, an optionally substituted cycloalkenyl group, an optionally substituted heterocyclic group, an optionally substituted amino group, an optionally substituted hydroxy group, an optionally substituted thiol group, a halogen atom (e.g., fluorine, chlorine, bromine, iodine) and oxo etc., and the hydrocarbon group is optionally substituted with arbitrary 1 to 5 (preferably 1 to 3) of these substituents at a substitutable position. Examples of the aryl group of the optionally substituted aryl group include a C₆₋₁₆ aryl group such as phenyl, naphthyl, anthryl, phenanthryl and acenaphthylenyl, inter alia, a C₆₋₁₀ aryl group such as phenyl, 1-naphthyl and 2-naphthyl is preferred. The substituent of the optionally substituted aryl group includes a C₁₋₃ alkoxy group (e.g., methoxy, ethoxy, propoxy, etc.), a halogen atom (e.g., fluorine, chlorine, bromine, iodine), a C₁₋₃ alkyl group (e.g., methyl, ethyl, propyl, etc.) and the like, and the aryl group is optionally substituted with arbitrary 1 to 2 of these substituents. Examples of the cycloalkyl group of the optionally substituted cycloalkyl group include a C₃₋₇ cycloalkyl group such as cyclopropyl, cyclobutyl, cyclopentyl, cyclohexyl and cycloheptyl. As for the substituent of the optionally substituted cycloalkyl group and the number of the substituents, the same kind and number as in the substituent for the aforementioned optionally substituted aryl group may be exemplified. Examples of the cycloalkenyl group of the optionally substituted cycloalkenyl group include a C₃₋₆ cycloalkenyl group such as cyclopropenyl, cyclobutenyl, cyclopentenyl and cyclohexenyl. As for the substituent of the optionally substituted cycloalkenyl group and the number of the substituents, the same kind and number as in the substituent for the aforementioned optionally substituted aryl group may be exemplified. A heterocyclic group of the optionally substituted heterocyclic group includes an aromatic heterocyclic group and a saturated or unsaturated non-aromatic heterocyclic group (aliphatic heterocyclic group) containing at least one and preferably 1 to 4 hetero atoms selected from oxygen, sulfur and nitrogen as a ring system constituent atom (ring atom), and an aromatic heterocyclic group is preferred. Examples of the aromatic heterocyclic group include a 5- to 6-membered aromatic monocyclic heterocyclic group (e.g., furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyridazinyl, pyrimidinyl, pyrazinyl, triazinyl, etc.) and an aromatic fused heterocyclic group in which 2 to 3 of 5- to 6-membered rings are fused (e.g., benzofuranyl, isobenzofuranyl, benzo[b]thienyl, indolyl, isoindolyl, 1H-indazolyl, benzimidazolyl, benzoxazolyl, 1,2-benzoisoxazolyl, benzothiazolyl, 1,2-benzoisothiazolyl, 1H-benzotriazolyl, quinolyl, isoquinolyl, cinnolyl, quinazolinyl, quinoxalinyl, phthalazinyl, naphthylizinyl, purinyl, pteridinyl, carbazolyl, α-carbolinyl, β-carbolinyl, γ-carbolinyl, acridinyl, phenoxazinyl, phenothiazinyl, phenazinyl, phenoxathinyl, thianthrenyl, phenanthridinyl, phenanthrolinyl, indolizinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl, etc.), inter alia, a 5- to 6-membered aromatic monocyclic heterocyclic group such as furyl, thienyl, indolyl, isoindolyl, pyrazinyl, pyridyl and pyrimidinyl is preferred. Examples of the non-aromatic heterocyclic group include a 4- to 8-membered non-aromatic heterocyclic group such as oxiranyl, azetidinyl, oxetanyl, thietanyl, pyrrolidinyl, tetrahydrofuryl, thiolanyl, piperidyl, tetrahydropyranyl, morpholinyl, thiomorpholinyl and piperazinyl. The optionally substituted heterocyclic group may have 1 to 4, preferably 1 to 2 substituents, and such substituents include C₁₋₃ alkyl group (e.g., methyl, ethyl, propyl, etc.) and the like. As the substituent in the optionally substituted amino group (including amino group, mono- or di-substituted amino group), the optionally substituted hydroxy group and the optionally substituted thiol group, a lower (C₁₋₃) alkyl (e.g., methyl, ethyl, propyl, etc.) and the like are exemplified. Further, when the hydrocarbon group in the optionally substituted hydrocarbon group represented by R₁ is an alicylcic hydrocarbon group or an aryl group, the substituent may be also a C₁₋₃ alkyl group (e.g., methyl, ethyl, propyl, etc.).

In addition, as described above, R₁ may have an oxo group as a substituent, and a carboxylic acid acyl group which is such a hydrocarbon group substituted with oxo is included in R₁. Examples thereof include an optionally substituted C₁₋₆ acyl group (e.g., formyl, acetyl, propionyl, butyryl, isobutyryl, valeryl, isovaleryl, pivaloyl, hexanoyl, dimethylacetyl, trimethylacetyl, etc.) and the like. Further, the acyl group may have 1 to 5 substituents at a substitutable position, and the substituent includes a halogen atom (e.g., fluorine, chlorine, bromine).

In the formulae (I) and (Ia), the “optionally substituted hydrocarbon group” represented by R₂ and R₃ may include the group descried as the “optionally substituted hydrocarbon group” represented by R₁. However, an alkyl group, an aryl group and substituents thereof may be the group as follows. That is, as for the alkyl group of the “optionally substituted alkyl group”, a C₁₋₆ lower alkyl group (e.g., methyl, ethyl, n-propyl, isopropyl, butyl, isobutyl, sec-butyl, tert-butyl, pentyl, isopentyl, neopentyl, hexyl, isohexyl, etc.) is exemplified, and preferably a C₁₋₄ alkyl group such as methyl, ethyl, propyl, isopropyl, butyl, tert-butyl is exemplified. For example, these optionally substituted alkyl group may have 1 to 4 substituents, and such substituents include a halogen atom (e.g., fluorine, chlorine, bromine, iodine), C₁₋₄ lower alkoxy group (e.g., methoxy, ethoxy, propoxy, isopropoxy, butoxy, tert-butoxy, etc.) and the like.

The “optionally substituted aryl group” includes monocyclic or fused polycyclic aromatic hydrocarbon group such as phenyl, naphthyl, anthryl, phenanthryl and acenaphthylenyl, and among them, phenyl is particularly preferred. The substituent of the “optionally substituted aryl group” includes a halogen atom (e.g., fluorine, chlorine, bromine, iodine etc.), optionally substituted lower alkyl group, optionally substituted lower alkoxy group, an optionally substituted hydroxy group, nitro and cyano, and may be substituted with the same or different 1 to 3 (preferably 1 to 2) of these substituents. Examples of the lower alkyl include a C₁₋₄ alkyl group such as methyl, ethyl, n-propyl, isopropyl, n-butyl, isobutyl, sec-butyl and tert-butyl, inter alia, methyl and ethyl is particularly preferred. Examples of the lower alkoxy include a C₁₋₄ alkoxy group such as methoxy, ethoxy, n-propoxy, isopropoxy, n-butoxy, isobutoxy, sec-butoxy, tert-butoxy, inter alia, methoxy and ethoxy is particularly preferred. The substituent of the optionally substituted lower alkyl and the optionally substituted lower alkoxy includes a halogen atom (e.g., fluorine, chlorine, bromine, iodine etc.), and may be substituted with 1 to 5 at an arbitrary substitutable position. Examples of the substituent in the optionally substituted hydroxy group include a lower (C₁₋₄) alkyl group (e.g., methyl, ethyl, propyl, isopropyl, butyl, tert-butyl etc.), a C₃₋₆ cycloalkyl group (e.g., cyclopropyl, cyclobutyl, cyclopentyl, cyclohexyl, etc.), a C₆₋₁₀ aryl group (e.g., phenyl, 1-naphthyl, 2-naphthyl, etc.) and a C₇₋₁₂ aralkyl group (e.g., benzyl, phenethyl, etc.). Further, these substituents may be combined together with the adjacent substituent to form a ring, and when the aryl group of the “optionally substituted aryl group” represented by R₂ and R₃ is a phenyl group, a group represented by

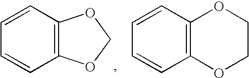

may be used, and furthermore, such groups may be substituted with 1 to 4 of lower (C₁₋₃) alkyl group (e.g., methyl, ethyl, propyl, isopropyl, etc.) and the like.

The heterocyclic group of the “optionally substituted heterocyclic group” represented by R₂ and R₃ includes the heterocyclic group described in detail for the “optionally substituted heterocyclic group” given as a substituent for the “optionally substituted hydrocarbon group” represented by R₁. Among those, 5- to 6-membered aromatic monocyclic heterocyclic ring such as furyl, thienyl, indolyl, isoindolyl, pyrazinyl, pyridyl, pyrimidyl and imidazolyl is particularly preferred. The substituent for the heterocyclic group includes C₁₋₃ alkyl (e.g., methyl, ethyl, propyl, etc.), and said heterocyclic ring may have 1 to 4 of such substituents.

Among the above, as for R₂ and R₃, an optionally substituted phenyl group is preferred, a substituted phenyl group is more preferred, and particularly, a phenyl group substituted with 1 to 3, preferably 1 to 2 of a halogen atom such as chlorine and bromine, lower (C₁₋₃) alkoxy or the like is preferred. Further, any one of R₂ and R₃ is preferably a hydrogen atom.

In the formula (I), the “group comprising an optionally esterified carboxyl group” represented by X′ includes an optionally esterified carboxyl group and a group having an optionally esterified carboxyl group. The optionally esterified carboxyl group includes the same group as that defined with respect to Y hereinafter.

The “group comprising an optionally substituted carbamoyl group” represented by X′ includes an optionally substituted carbamoyl group and a group having an optionally substituted carbamoyl group. The optionally substituted carbamoyl group includes the same group as that defined with respect to Y hereinafter.

The “group comprising an optionally substituted hydroxy group” represented by X′ includes an optionally substituted hydroxy group and a group having an optionally substituted hydroxy group. The optionally substituted hydroxy group includes the same group as that defined with respect to Y hereinafter.

The “group comprising an optionally substituted amino group” represented by X′ includes an optionally substituted amino group and a group having an optionally substituted amino group. The optionally substituted amino group includes the same group as that defined with respect to Y hereinafter.

The “group comprising an optionally substituted heterocyclic residue having a hydrogen atom which can be deprotonated” represented by X′ includes an optionally substituted heterocyclic residue having a hydrogen atom which can be deprotonated (i.e., having an active proton) and a group having an optionally substituted heterocyclic residue having a hydrogen atom which can be deprotonated. The optionally substituted heterocyclic residue having a hydrogen atom which can be deprotonated includes the same group as that defined with respect to Y hereinafter.

X′ includes a group represented by the formula (a):

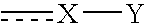

wherein, X is a bond, or divalent or trivalent atomic chain, Y is an optionally esterified carboxyl group, an optionally substituted carbamoyl group, an optionally substituted hydroxy group, an optionally substituted amino group, or an optionally substituted heterocyclic residue having a hydrogen atom which can be deprotonated, and the dotted line is a single or double bond.

In the formula (a), the “divalent atomic chain” represented by X may be any divalent chain having preferably 1 to 7, and more preferably 1 to 4 of atoms composing the linear portion, and may have a side chain.

Example thereof includes a group represented by

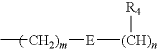

wherein, m and n represent an integer of 0, 1, 2 or 3, independently, E represents a bond or an oxygen atom, a sulfur atom, sulfoxide, sulfone, —N(R₅)—, —NHCO—, —CON(R₆)— or —NHCONH—. Herein, R₄ and R₆ represent a hydrogen atom, an optionally substituted lower alkyl group, an optionally substituted aralkyl group or an optionally substituted phenyl group. In addition, R₅ represents a hydrogen atom, a lower alkyl group, an aralkyl group or an acyl group.

The alkyl group of the “optionally substituted lower alkyl group” represented by R₄ and R₆ includes a C₁₋₆ linear or branched lower alkyl group (e.g., methyl, ethyl, n-propyl, isopropyl, n-butyl, isobutyl, tert-butyl, n-pentyl, isopentyl, neopentyl, etc.). The optionally substituted lower alkyl group may have 1 to 4, preferably 1 to 2 substituents, and examples of such substituents include an aromatic heterocyclic group (e.g., 5- to 6-membered aromatic heterocyclic ring containing 1 to 4 hetero atoms of N, O and S such as furyl, thienyl, indolyl, isoindolyl, pyrazinyl, pyridyl, pyrimidyl and imidazolyl), an optionally substituted amino group, an optionally substituted hydroxy group, an optionally substituted thiol group, an optionally esterified carboxyl group and a halogen atom (e.g., fluorine, chlorine, bromine, iodine). The substituent in the optionally substituted amino group (including amino group, mono- or di-substituted amino group), an optionally substituted hydroxy group and an optionally substituted thiol group includes lower (C₁₋₃) alkyl (e.g., methyl, ethyl, propyl, etc.). Examples of the optionally esterified carboxyl group include C₂₋₅ alkoxycarbonyl such as methoxycarbonyl ethoxycarbonyl, propoxycarbonyl, phenoxycarbonyl and 1-naphthoxycarbonyl, and C₇₋₁₁ aryloxycarbonyl, and preferably, methoxycarbonyl, ethoxycarbonyl and propoxycarbonyl are exemplified.

The aralkyl group of the “optionally substituted aralkyl group” represented by R₄ and R₆ includes a C₇-C₁₅ aralkyl group such as benzyl, naphthylmethyl, phenylpropyl and phenylbutyl. The optionally substituted aralkyl group may have 1 to 4, preferably 1 to 2 substituents, and such substituents include a halogen atom (e.g., fluorine, chlorine, bromine, iodine), a C₁₋₃ alkoxy group (e.g., methoxy, ethoxy, propoxy group), a hydroxy group, an amino group, a carboxyl group, a sulfhydryl group etc.

The substituent of the “optionally substituted phenyl group” represented by R₄ and R₆ includes a halogen atom (e.g., fluorine, chlorine, bromine, iodine), a C₁₋₃ alkoxy (e.g., methoxy, ethoxy, propoxy, etc.), C₁₋₃ alkyl (e.g., methyl, ethyl, propyl).

Provided that, R₄ may be different in every methylene chain.

In addition, examples of the “lower alkyl group” and the “aralkyl group” represented by R₅ include a C₁₋₄ lower alkyl group (e.g., methyl, ethyl, propyl, butyl, tert-butyl, etc.), a C₇₋₁₅ aralkyl group (e.g., benzyl, phenethyl, phenylpropyl, phenylbutyl, naphthylmethyl, etc.), respectively.

Examples of the “acyl group” represented by R₅ include a lower (C₁₋₆) alkanoyl group (e.g., formyl, acetyl, propionyl, butyryl, isobutyryl, valeryl, isovaleryl, pivaloyl, hexanoyl, etc.), a lower (C₃₋₇) alkenoyl group (e.g., acryloyl, methacryloyl, crotonoyl, isocrotonoyl, etc.), a C₄₋₇ cycloalkanecarbonyl group (e.g., a cyclopropanecarbonyl group, a cyclobutanecarbonyl group, a cyclopentanecarbonyl group, a cyclohexanecarbonyl group, etc.), a lower (C₁₋₄) alkanesulfonyl group (e.g., mesyl, ethanesulfonyl, propanesulfonyl, etc.), a C₇₋₁₄ aroyl group (e.g., benzoyl, p-toluoyl, 1-naphthoyl, 2-naphthoyl, etc.), a C₆₋₁₀ aryl lower (C₂₋₄) alkanoyl group (e.g., phenylacetyl, phenylpropionyl, hydroatropoyl, phenylbutyryl, etc.), a C₆₋₁₀ aryl lower (C₃₋₅) alkenoyl group (e.g., cinnamoyl, atropoyl, etc.), a C₆₋₁₀ arenesulfonyl group (e.g., benzenesulfonyl, a p-toluenesulfonyl group, etc.).

Further, X may be a carbon chain having a double bond or -L-CH(OH)— (L represents a bond or a linear or branched alkylene chain). Examples of the “carbon chain having a double bond” include a carbon chain having, preferably 1 to 7, more preferably 1 to 4 of carbon atoms constituting the linear portion, and may also have a side chain. The double bond in the carbon chain is contained in any one or both of a linear portion and a branched portion, and preferably contained in the linear portion. Further, the number of double bonds contained in the carbon chain is not particularly limited, if possible, but 1 to 2 is preferred.

Examples of the carbon chain having double bond include methine, vinylene, propenylene, butenylene, butadienylene, methylpropenylene, ethylpropenylene, propylpropenylene, methylbutenylene, ethylbutenylene, propylbutenylene, methylbutadienylene, ethylbutadienylene, propylbutadienylene, pentenylene, hexenylene, heptenylene, pentadienylene, hexadienylene, heptadienylene and the like, and preferably, methine, vinylene, propenylene, butenylene and butadienylene are exemplified. Herein, when the carbon chain is trivalent, the carbon chain forms a double bond with a substitutable carbon atom on the ring of ring J′.

Examples of the “linear or branched alkylene chain” represented by L include a linear or branched C₁₋₆ alkylene chain, for example, a divalent group such as methylene, ethylene, trimethylene, tetramethylene, pentamethylene, hexamethylene, heptamethylene, propylene, ethylmethylene, ethylethylene, propylethylene, butylethylene, methyltetramethylene and methyltrimethylene, and preferably, a C₁₋₃ chain such as methylene, ethylene, trimethylene and propylene are exemplified.

Among these, X′ is preferably a group represented by the formula (b):

—X₁—Y

wherein X₁ represents a bond or divalent atomic chain, Y represents an optionally esterified carboxyl group, an optionally substituted carbamoyl group, an optionally substituted hydroxy group, an optionally substituted amino group or an optionally substituted heterocyclic group having a hydrogen atom which can be deprotonated.

In the formula (b), as for the divalent atomic chain represented by X₁, the same as in the divalent atomic chain defined with respect to the aforementioned X may be exemplified.

In the formulae (a) and (b), the “divalent atomic chain” represented by X or X₁ includes a linear or branched alkylene chain having preferably 1 to 7 (more preferably 1 to 4) of carbon atoms constituting the linear portion. Examples of the alkylene chain include a divalent group such as methylene, ethylene, trimethylene, tetramethylene, pentamethylene, hexamethylene, heptamethylene, propylene, ethylmethylene, ethylethylene, propylethylene, butylethylene, methyltetramethylene and methyltrimethylene, and preferably, a C₁₋₄ chain such as methylene, ethylene, trimethylene and propylene is exemplified.

In the formulae (a) and (b), the “optionally esterified carboxyl group” represented by Y includes a C₂₋₇ lower alkoxycarbonyl (e.g., methoxycarbonyl, ethoxycarbonyl, propoxycarbonyl, isopropoxycarbonyl, butoxycarbonyl, tert-butoxycarbonyl, sec-butoxycarbonyl, pentyloxycarbonyl, isopentyloxycarbonyl, neopentyloxycarbonyl, etc.), C₇₋₁₄ aryloxycarbonyl (e.g., phenoxycarbonyl, 1-naphthoxycarbonyl) and C₈₋₁₂ aralkyloxycarbonyl (e.g., benzyloxycarbonyl, etc.). Among these, a carboxyl group, methoxycarbonyl, and ethoxycarbonyl are preferred.

The substituent of the “optionally substituted carbamoyl group” represented by Y includes an optionally substituted lower (C₁₋₆) alkyl (e.g., methyl, ethyl, n-propyl, isopropyl, butyl, isobutyl, sec-butyl, tert-butyl, pentyl, isopentyl, neopentyl, hexyl, isohexyl, etc.), an optionally substituted C₃₋₆ cycloalkyl (e.g., cyclopropyl, cyclobutyl, cyclopentyl, cyclohexyl, etc.), an optionally substituted C₆₋₁₄ aryl group (e.g., phenyl, 1-naphthyl, 2-naphthyl, etc.) and an optionally substituted C₇₋₁₁ aralkyl group (e.g., benzyl, phenethyl, etc.), and the carbamoyl group may be substituted with the same or different 1 to 2 of these substituents. The substituent in the optionally substituted lower (C₁₋₆) alkyl and optionally substituted C₃₋₆ cycloalkyl includes a carboxyl group optionally esterified with lower (C₁₋₅) alkyl (e.g., methyl, ethyl, propyl, isopropyl, butyl, tert-butyl, pentyl, isopentyl, neopentyl), a 5- to 6-membered aromatic heterocyclic group containing 1 to 4 hetero atoms (e.g., furyl, thienyl, indolyl, isoindolyl, pyrazinyl, pyridyl, pyrimidyl, imidazolyl, etc.), an amino group, a hydroxy group and a phenyl group, and the same or different 1 to 3 of these substituents may substitute. The substituent of the optionally substituted aryl group and the optionally substituted aralkyl group includes a halogen atom (e.g., fluorine, chlorine, bromine, iodine), and carboxyl group optionally esterified with a lower (C₁₋₄) alkyl group (e.g., methyl, ethyl, propyl, isopropyl, butyl, tert-butyl, etc.). In addition, in the optionally substituted carbamoyl group, the two substituents on the nitrogen atoms may be combined together with the nitrogen atoms to form a cyclic amino group, and examples of such cyclic amino group include 1-azetidinyl, 1-pyrrolidinyl, piperidino, morpholino, 1-piperazinyl and the like. Further, the cyclic amino group may also have a substituent.

The substituent of the “optionally substituted hydroxy group” represented by Y includes, for example, lower (C₁₋₄) alkyl (e.g., methyl, ethyl, propyl, isopropyl, butyl, tert-butyl, etc.), a C₃₋₆ cycloalkyl group (e.g., cyclopropyl, cyclobutyl, cyclopentyl, cyclohexyl, etc.), an optionally substituted C₆₋₁₀ aryl group (e.g., phenyl, 1-naphthyl, 2-naphthyl, etc.) and an optionally substituted C₇₋₁₁ aralkyl group (e.g., benzyl, phenethyl, etc.). The substituent of the optionally substituted aryl group and the optionally substituted aralkyl group includes a halogen atom (e.g., fluorine, chlorine, bromine, iodine), carboxyl group optionally esterified with a lower (C₁₋₄) alkyl group (e.g., methyl, ethyl, propyl, isopropyl, butyl, tert-butyl, etc.), and the like.

The “optionally substituted amino group” represented by Y includes a mono-substituted and di-substituted amino group, and examples of such substituent include lower (C₁₋₄) alkyl (e.g., methyl, ethyl, propyl, isopropyl, butyl, tert-butyl, etc.), a C₃₋₆ cycloalkyl group (e.g., cyclopropyl, cyclobutyl, cyclopentyl, cyclohexyl, etc.), an optionally substituted C₆₋₁₀ aryl group (e.g., phenyl, 1-naphthyl, 2-naphthyl, etc.), an optionally substituted C₇₋₁₁ aralkyl group (e.g., benzyl, phenethyl, etc.) and the like. The substituent of the optionally substituted aryl group and the optionally substituted aralkyl group includes a halogen atom (e.g., fluorine, chlorine, bromine, iodine), carboxyl group optionally esterified with a lower (C₁₋₄) alkyl group (e.g., methyl, ethyl, propyl, isopropyl, butyl, tert-butyl, etc.) and the like, and 1 to 4, preferably 1 to 2 of these substituents may be possessed. In addition, two of the substituents on the nitrogen atom may be combined together with the nitrogen atom to form a cyclic amino group, and examples of such cyclic amino group include 1-azetidinyl, 1-pyrrolidinyl, piperidino, morpholino, 1-piperazinyl. In addition, the cyclic amino group may also have a further substituent.

The heterocyclic residue of the “optionally substituted heterocyclic group having a hydrogen atom which can be deprotonated” represented by Y includes a 5- to 7-membered (preferably 5-membered) monocyclic heterocyclic residue having at least one selected from N, S and O (preferably a nitrogen-containing heterocyclic residue), which has a hydrogen atom that can eliminate to form a proton. Examples thereof include tetrazol-5-yl or a group represented by the formula:

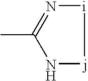

wherein, i represents —O— or —S—, j represents >C═O, >C═S or >S(O)₂, (among these, 2,5-dihydro-5-oxo-1,2,4-oxadiazol-3-yl, 2,5-dihydro-5-thioxo-1,2,4-oxadiazol-3-yl and 2,5-dihydro-5-oxo-1,2,4-thiadiazol-3-yl are preferred).

The above heterocyclic residue may be protected with an optionally substituted lower alkyl group (preferably C₁₋₄ alkyl) or an acyl group. Examples of the optionally substituted lower alkyl group include C₁₋₄ alkyl optionally substituted with 1) phenyl optionally substituted with C₁₋₃ alkyl, nitro or C₁₋₃ alkoxy or 2) C₁₋₃ alkoxy (e.g., methyl, triphenylmethyl, methoxymethyl, ethoxymethyl, p-methoxybenzyl, p-nitrobenzyl, etc.). Examples of the acyl group include lower (C₂₋₅) alkanoyl, benzoyl and the like.

Among these, X′ is preferably an alkyl group substituted with an optionally esterified carboxyl group, an alkyl group substituted with an optionally substituted heterocyclic residue having a hydrogen which can be deprotonated or an alkyl group substituted with an optionally substituted carbamoyl group.

In the formula (I), the heterocyclic ring represented by Ring A includes a heterocyclic group described in detail with respect to the substituent of the hydrocarbon group represented by R₁. Among them, a group represented below is preferred.

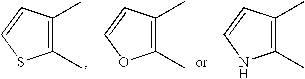

The substituent of the “optionally substituted benzene ring” and “optionally substituted heterocyclic ring” represented by Ring A includes a halogen atom (e.g., fluorine, chlorine, bromine, iodine), an optionally substituted C₁₋₄ lower alkyl group (e.g., methyl, ethyl, propyl, butyl, tert-butyl, etc.), an optionally substituted C₁₋₄ lower alkoxy group (e.g., methoxy, ethoxy, propoxy, isopropoxy, butoxy, tert-butoxy, etc.), a hydroxy group, a nitro group and cyano. Ring A may have 1 to 3, preferably 1 to 2 of these substituents. Further, these substituents may be combined together with the adjacent substituents to form a ring. The substituent of the optionally substituted lower alkyl group and the optionally substituted lower alkoxy group includes a halogen atom (e.g., fluorine, chlorine, bromine, iodine) and the like, and 1 to 3 of substituents may be present at an arbitrary position. Ring A is preferably substituted with methoxy or a chlorine atom, and Ring A substituted with a chlorine atom is particularly preferred.

In the formula (Ia), the substituent of the “optionally substituted benzene ring” represented by Ring B includes a halogen atom (e.g., fluorine, chlorine, bromine, iodine), an optionally substituted C₁₋₄ lower alkyl group (e.g., methyl, ethyl, propyl, butyl, tert-butyl, etc.), an optionally substituted C₁₋₄ lower alkoxy group (e.g., methoxy, ethoxy, propoxy, isopropoxy, butoxy, tert-butoxy, etc.), a hydroxy group, a nitro group and cyano. Ring B may have 1 to 3, preferably 1 to 2 of these substituents. Further, these substituents may be combined together with the adjacent substituents to form a ring. The substituent of the optionally substituted lower alkyl group and the optionally substituted lower alkoxy group includes a halogen atom (e.g., fluorine, chlorine, bromine, iodine) and the like, and 1 to 3 of substituents may be present at an arbitrary position. Ring B is preferably substituted with methoxy or a chlorine atom, and Ring B substituted with a chlorine atom is particularly preferred.

In the formula (I), the heterocyclic ring in the “7- to 8-membered heterocyclic ring containing 3 or less hetero atoms as ring constituent atoms” represented by the ring J′ includes, for example, a saturated or unsaturated 7- or 8-membered heterocyclic ring containing at least one selected from O, S(O)_{q} (q represents 0, 1 or 2) and N. However, the hetero atoms in the atoms constituting the ring of said heterocyclic ring (ring constituent atom) are three or less.

Further, Ring J′ may have 1 to 2 substituents at a substitutable position in addition to a group represented by R₁, R₂, R₃ and X′. When the substituent is attached to a nitrogen atom on Ring J′, examples of the substituent include an alkyl group (e.g., C₁₋₆ alkyl such as methyl, ethyl, n-propyl, isopropyl, n-butyl, isobutyl, tert-butyl, n-pentyl, isopentyl, neopentyl, etc.), an acyl group (e.g., C₁₋₄ acyl group such as formyl, acetyl, propionyl, butyroyl, etc.). The alkyl group or acyl group may further be substituted with 1 to 5 of halogen atoms (e.g., fluorine, chlorine, bromine, iodine). Further, when the substituent is attached to a carbon atom on the Ring J′, examples of the substituent include oxo, thioxo, an optionally substituted hydroxy group and an optionally substituted amino group. As for the optionally substituted hydroxy group and the optionally substituted amino group, the same as in the “optionally substituted hydroxy group” and the “optionally substituted amino group” defined as Y above may be exemplified.

Ring J′ is preferably substituted with oxo or thioxo at a substitutable position in addition to the group represented by R₁, R₂, R₃ and X′.

Examples of a fused ring comprising Ring A and ring J′ include

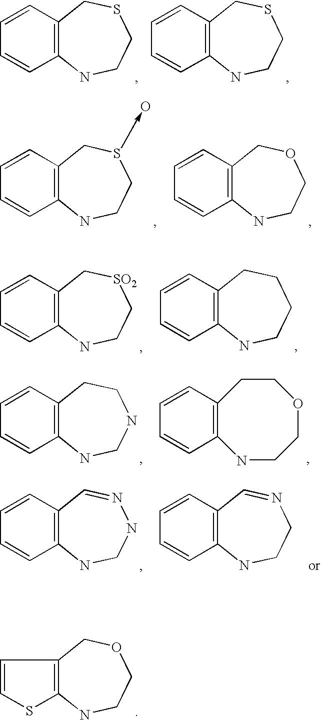

The formula (I) is preferably a group represented by the formula (I′)

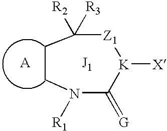

wherein, R₁, R₂, R₃, X′ and Ring A are as defined above, and

Ring J₁ represents a 7-membered heterocyclic ring, Z₁ represents —N(R₇)— (R₇ represents a hydrogen atom, an alkyl group or an acyl group), —S(O)_{q} (q represents 0, 1 or 2), —CH₂— or —O—, K represents C or N, and G represents O or S.

In the formula (I′) above, the alkyl group represented by R₇ includes a C₁₋₆ linear or branched lower alkyl group (e.g., methyl, ethyl, n-propyl, isopropyl, n-butyl, isobutyl, tert-butyl, n-pentyl, isopentyl, neopentyl, etc.), which may be substituted with 1 to 5 of halogen atoms (e.g., fluorine, chlorine, bromine, iodine).

Examples of the acyl group represented by R₇ include a C₁₋₄ acyl group (e.g., formyl, acetyl, propionyl, butyroyl, etc.), which may be substituted with 1 to 5 of halogen atoms (e.g., fluorine, chlorine, bromine, iodine).

In the formula (I′), Z₁ is preferably S(O)_{q} (q represents 0, 1 or 2) or O. Further, K is preferably C and G is preferably O.

As for the formula (I′), a compound represented by the formula (I″)

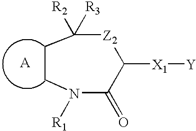

wherein, R₁, R₂, R₃, X₁, Y and Ring A are as defined above, and Z₂ represents S(O)_{q} (q represent 0, 1 or 2) or O, is more preferred.

The compound represented by the formula (I) is preferably the compound represented by the formula (Ia)

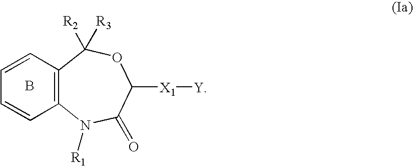

The compound of formula (Ia) may be also a compound represented by the formula (Ia′)

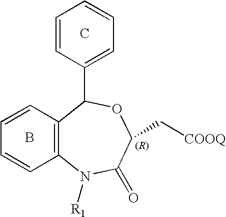

wherein, R₁ and Ring B are as defined above, and Q represents a hydrogen atom or a metal ion, Ring C represents an optionally substituted phenyl group. In the formula, the substituents at 3- and 5-position represent trans which faces the opposite direction relative to the plane of the 7-membered ring, and (R) represents R-configuration.

In the formula (Ia′), the metal ion represented by Q includes a sodium ion, a potassium ion, a calcium ion, an aluminum ion and the like, inter alia, a sodium ion and a potassium ion are preferred.

The substituent of the “optionally substituted phenyl group” represented by Ring C includes the same group as the substituent of the “optionally substituted aryl group” described as an example of the “optionally substituted hydrocarbon group” defined with respect to R₂ and R₃ above.

Examples of the salt of the compound represented by the formula (I) include pharmacologically acceptable salts such as an inorganic salt such as hydrochloride, hydrobromide, sulfate, nitrate and phosphate, an organic acid salt such as acetate, tartrate, citrate, fumarate, maleate, toluenesulfonate and methanesulfonate, a metal salt such as sodium salt, potassium salt, calcium salt and aluminum salt, and a salt with base such as triethylamine salt, guanidine salt, ammonium salt, hydrazine salt, quinine salt and cinchonine salt. Among these, a sodium salt is preferred.

Specific examples of the compound represented by the formula (I) includes below:
(3R,5S)-7-cyano-5-(2,3-dimethoxyphenyl)-1-neopentyl-2-oxo-1,2,3,5-tetrahydro-4,1-benzoxazepine-3-acetic acid,
(3R,5S)-7-cyano-5-(2,4-dimethoxyphenyl)-1-neopentyl-2-oxo-1,2,3,5-tetrahydro-4,1-benzoxazepine-3-acetic acid,
(3R,5S)-7-cyano-5-(2,3-methylenedioxyphenyl)-1-neopentyl-2-oxo-1,2,3,5-tetrahydro-4,1-benzoxazepine-3-acetic acid,
(3R,5S)-7-cyano-5-(2,3-ethylenedioxyphenyl)-1-neopentyl-2-oxo-1,2,3,5-tetrahydro-4,1-benzoxazepine-3-acetic acid,
(3R,5S)-7-cyano-5-(2,3-dimethoxyphenyl)-1-isobutyl-2-oxo-1,2,3,5-tetrahydro-4,1-benzoxazepine-3-acetic acid,
(3R,5S)-7-cyano-5-(2,4-dimethoxyphenyl)-1-isobutyl-2-oxo-1,2,3,5-tetrahydro-4,1-benzoxazepine-3-acetic acid,
(3R,5S)-7-cyano-5-(2,3-methylenedioxyphenyl)-1-isobutyl-2-oxo-1,2,3,5-tetrahydro-4,1-benzoxazepine-3-acetic acid,
(3R,5S)-7-cyano-5-(2,3-ethylenedioxyphenyl)-1-isobutyl-2-oxo-1,2,3,5-tetrahydro-4,1-benzoxazepine-3-acetic acid,
(3R,5S)-7-chloro-5-(2,3-dimethoxyphenyl)-1-neopentyl-2-oxo-1,2,3,5-tetrahydro-4,1-benzoxazepine-3-acetic acid,
(3R,5S)-7-chloro-5-(2,4-dimethoxyphenyl)-1-neopentyl-2-oxo-1,2,3,5-tetrahydro-4,1-benzoxazepine-3-acetic acid,
(3R,5S)-7-chloro-5-(2,3-methylenedioxyphenyl)-1-neopentyl-2-oxo-1,2,3,5-tetrahydro-4,1-benzoxazepine-3-acetic acid,
(3R,5S)-7-chloro-5-(2,3-ethylenedioxyphenyl)-1-neopentyl-2-oxo-1,2,3,5-tetrahydro-4,1-benzoxazepine-3-acetic acid,
(3R,5S)-7-chloro-5-(2,3-dimethoxyphenyl)-1-isobutyl-2-oxo-1,2,3,5-tetrahydro-4,1-benzoxazepine-3-acetic acid,
(3R,5S)-7-chloro-5-(2,4-dimethoxyphenyl)-1-isobutyl-2-oxo-1,2,3,5-tetrahydro-4,1-benzoxazepine-3-acetic acid,
(3R,5S)-7-chloro-5-(2,3-methylenedioxyphenyl)-1-isobutyl-2-oxo-1,2,3,5-tetrahydro-4,1-benzoxazepine-3-acetic acid,
(3R,5S)-7-chloro-5-(2,3-ethylenedioxyphenyl)-1-isobutyl-2-oxo-1,2,3,5-tetrahydro-4,1-benzoxazepine-3-acetic acid,
(3R,5S)-7-cyano-5-(2,3-dimethoxyphenyl)-1-neopentyl-2-oxo-1,2,3,5-tetrahydro-4,1-benzothiazepine-3-acetic acid,
(3R,5S)-7-cyano-5-(2,4-dimethoxyphenyl)-1-neopentyl-2-oxo-1,2,3,5-tetrahydro-4,1-benzothiazepine-3-acetic acid,
(3R,5S)-7-cyano-5-(2,3-methylenedioxyphenyl)-1-neopentyl-2-oxo-1,2,3,5-tetrahydro-4,1-benzothiazepine-3-acetic acid,
(3R,5S)-7-cyano-5-(2,3-ethylenedioxyphenyl)-1-neopentyl-2-oxo-1,2,3,5-tetrahydro-4,1-benzothiazepine-3-acetic acid,
(3R,5S)-7-cyano-5-(2,3-dimethoxyphenyl)-1-isobutyl-2-oxo-1,2,3,5-tetrahydro-4,1-benzothiazepine-3-acetic acid,
(3R,5S)-7-cyano-5-(2,4-dimethoxyphenyl)-1-isobutyl-2-oxo-1,2,3,5-tetrahydro-4,1-benzothiazepine-3-acetic acid,
(3R,5S)-7-cyano-5-(2,3-methylenedioxyphenyl)-1-isobutyl-2-oxo-1,2,3,5-tetrahydro-4,1-benzothiazepine-3-acetic acid,
(3R,5S)-7-cyano-5-(2,3-ethylenedioxyphenyl)-1-isobutyl-2-oxo-1,2,3,5-tetrahydro-4,1-benzothiazepine-3-acetic acid,
(3R,5S)-7-chloro-5-(2,3-dimethoxyphenyl)-1-neopentyl-2-oxo-1,2,3,5-tetrahydro-4,1-benzothiazepine-3-acetic acid,
(3R,5S)-7-chloro-5-(2,4-dimethoxyphenyl)-1-neopentyl-2-oxo-1,2,3,5-tetrahydro-4,1-benzothiazepine-3-acetic acid,
(3R,5S)-7-chloro-5-(2,3-methylenedioxyphenyl)-1-neopentyl-2-oxo-1,2,3,5-tetrahydro-4,1-benzothiazepine-3-acetic acid,
(3R,5S)-7-chloro-5-(2,3-ethylenedioxyphenyl)-1-neopentyl-2-oxo-1,2,3,5-tetrahydro-4,1-benzothiazepine-3-acetic acid,
(3R,5S)-7-chloro-5-(2,3-dimethoxyphenyl)-1-isobutyl-2-oxo-1,2,3,5-tetrahydro-4,1-benzothiazepine-3-acetic acid,
(3R,5S)-7-chloro-5-(2,4-dimethoxyphenyl)-1-isobutyl-2-oxo-1,2,3,5-tetrahydro-4,1-benzothiazepine-3-acetic acid,
(3R,5S)-7-chloro-5-(2,3-methylenedioxyphenyl)-1-isobutyl-2-oxo-1,2,3,5-tetrahydro-4,1-benzothiazepine-3-acetic acid,
(3R,5S)-7-chloro-5-(2,3-ethylenedioxyphenyl)-1-isobutyl-2-oxo-1,2,3,5-tetrahydro-4,1-benzothiazepine-3-acetic acid,
(3R,5S)-7-cyano-5-(2-chlorophenyl)-1-neopentyl-2-oxo-1,2,3,5-tetrahydro-4,1-benzoxazepine-3-acetic acid,
(3R,5S)-7-cyano-5-(2-chlorophenyl)-1-isobutyl-2-oxo-1,2,3,5-tetrahydro-4,1-benzoxazepine-3-acetic acid,
(3R,5S)-7-chloro-5-(2-chlorophenyl)-1-neopentyl-2-oxo-1,2,3,5-tetrahydro-4,1-benzoxazepine-3-acetic acid,
(3R,5S)-7-chloro-5-(2-chlorophenyl)-1-isobutyl-2-oxo-1,2,3,5-tetrahydro-4,1-benzoxazepine-3-acetic acid,
(3R,5S)-7-cyano-5-(2-chlorophenyl)-1-neopentyl-2-oxo-1,2,3,5-tetrahydro-4,1-benzothiazepine-3-acetic acid,
(3R,5S)-7-cyano-5-(2-chlorophenyl)-1-isobutyl-2-oxo-1,2,3,5-tetrahydro-4,1-benzothiazepine-3-acetic acid,
(3R,5S)-7-chloro-5-(2-chlorophenyl)-1-neopentyl-2-oxo-1,2,3,5-tetrahydro-4,1-benzothiazepine-3-acetic acid,
(3R,5S)-7-chloro-5-(2-chlorophenyl)-1-isobutyl-2-oxo-1,2,3,5-tetrahydro-4,1-benzothiazepine-3-acetic acid,
(3R,5S)-7-chloro-5-(4-ethoxy-2-methoxyphenyl)-1-neopentyl-2-oxo-1,2,3,5-tetrahydro-4,1-benzoxazepine-3-acetic acid,
(3R)-7-chloro-5-(2-chlorophenyl)-2,3-dihydro-1-isobutyl-2-oxo-1H-1,4-benzodiazepine-3-acetic acid,
(3R,5S)-7-chloro-5-(2-chlorophenyl)-2,3,4,5-tetrahydro-1-isobutyl-2-oxo-1H-1,4-benzodiazepine-3-acetic acid,
N-[[(3R,5S)-7-chloro-5-(2-chlorophenyl)-1-neopentyl-2-oxo-1,2,3,5-tetrahydro-4,1-benzothiazepine-3-yl]-acetyl]glycine,
(3R,5S)-7-chloro-5-(2-chlorophenyl)-3-dimethylaminocarbonylmethyl-1-neopentyl-2-oxo-1,2,3,5-tetrahydro-4,1-benzothiazepine,
7-chloro-5-(2-chlorophenyl)-1-isobutyl-2-oxo-2,3,4,5-tetrahydro-1H-[1]-benzazepine-3-acetic acid,
(3R,5S)-7-chloro-5-(2-chlorophenyl)-1-neopentyl-1,2,3,5-tetrahydro-2-thioxo-4,1-benzoxazepine-3-acetic acid,
(3R,5S)-7-chloro-5-(2-chlorophenyl)-1-neopentyl-2-oxo-1,2,3,5-tetrahydro-4,1-thieno[2,3-e]oxazepine-3-acetic acid,
(3R,5S)-7-chloro-5-(2-methoxyphenyl)-1-neopentyl-2-oxo-1,2,3,5-tetrahydro-4,1-thieno[2,3-e]oxazepine-3-acetic acid,
(3R,5S)-7-chloro-1-isobutyl-5-(2-methoxyphenyl)-2-oxo-1,2,3,5-tetrahydro-4,1-thieno[2,3-e]oxazepine-3-acetic acid,
(3R,5S)-7-chloro-5-(3-hydroxy-2-methoxyphenyl)-1-neopentyl-2-oxo-1,2,3,5-tetrahydro-4,1-benzothiazepine-3-acetic acid,
(3R,5S)-7-chloro-5-(4-hydroxy-2-methoxyphenyl)-1-neopentyl-2-oxo-1,2,3,5-tetrahydro-4,1-benzothiazepine-3-acetic acid,
(3R,5S)-7-chloro-5-(3-hydroxy-2-methoxyphenyl)-1-isobutyl-2-oxo-1,2,3,5-tetrahydro-4,1-benzothiazepine-3-acetic acid,
(3R,5S)-7-chloro-5-(4-hydroxy-2-methoxyphenyl)-1-isobutyl-2-oxo-1,2,3,5-tetrahydro-4,1-benzothiazepine-3-acetic acid,
(3R,5S)-7-chloro-5-(3-ethoxy-2-methoxyphenyl)-1-neopentyl-2-oxo-1,2,3,5-tetrahydro-4,1-benzothiazepine-3-acetic acid,
(3R,5S)-7-chloro-5-(4-ethoxy-2-methoxyphenyl)-1-neopentyl-2-oxo-1,2,3,5-tetrahydro-4,1-benzothiazepine-3-acetic acid,
(3R,5S)-7-chloro-5-(3-ethoxy-2-methoxyphenyl)-1-isobutyl-2-oxo-1,2,3,5-tetrahydro-4,1-benzothiazepine-3-acetic acid,
(3R,5S)-7-chloro-5-(4-ethoxy-2-methoxyphenyl)-1-isobutyl-2-oxo-1,2,3,5-tetrahydro-4,1-benzothiazepine-3-acetic acid,
(3R,5S)-7-chloro-5-(2-chloro-3-methoxyphenyl)-1-neopentyl-2-oxo-1,2,3,5-tetrahydro-4,1-benzothiazepine-3-acetic acid,
(3R,5S)-7-chloro-5-(2-chloro-4-methoxyphenyl)-1-neopentyl-2-oxo-1,2,3,5-tetrahydro-4,1-benzothiazepine-3-acetic acid,
(3R,5S)-7-chloro-5-(2-chloro-3-methoxyphenyl)-1-isobutyl-2-oxo-1,2,3,5-tetrahydro-4,1-benzothiazepine-3-acetic acid,
(3R,5S)-7-chloro-5-(2-chloro-4-methoxyphenyl)-1-isobutyl-2-oxo-1,2,3,5-tetrahydro-4,1-benzothiazepine-3-acetic acid,
(3R,5S)-7-chloro-5-(2-chloro-3-hydroxyphenyl)-1-neopentyl-2-oxo-1,2,3,5-tetrahydro-4,1-benzothiazepine-3-acetic acid,
(3R,5S)-7-chloro-5-(2-chloro-4-hydroxyphenyl)-1-neopentyl-2-oxo-1,2,3,5-tetrahydro-4,1-benzothiazepine-3-acetic acid,
(3R,5S)-7-chloro-5-(2-chloro-3-hydroxyphenyl)-1-isobutyl-2-oxo-1,2,3,5-tetrahydro-4,1-benzothiazepin-3-acetic acid,
(3R,5S)-7-chloro-5-(2-chloro-4-hydroxyphenyl)-1-isobutyl-2-oxo-1,2,3,5-tetrahydro-4,1-benzothiazepine-3-acetic acid;
   and salts thereof.

The compounds represented by the formula (I) and the salts thereof [hereinafter, sometimes, abbreviated as Compound (I) including salts] are disclosed in, for example, EP-A-567026, WO95/21834 (Japanese Patent Application No. 6-15531), EP-A-645377 (Japanese Patent Application No. 6-229159), EP-A-645378 (Japanese Patent Application No. 6-229160), and can be prepared according to the disclosure of these publications.

The compound represented by the formula (I) is preferably the compound represented by the formula (Ib):

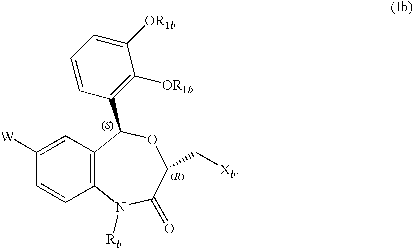

Preferable examples of the compound represented by the formula (Ib) include:

the compound wherein R_{b} is a C₁₋₆ alkyl group which may have 1 to 3 substituents selected from hydroxy group, acetyloxy, propionyloxy, t-butoxycarbonyloxy, palmitoyloxy, dimethylaminoacetyloxy and 2-aminopropionyloxy;
the compound wherein R_{b} is a branched C₃₋₆ alkyl group which may have 1 to 3 substituents selected from hydroxy group, acetyloxy, propionyloxy, t-butoxycarbonyloxy, palmitoyloxy, dimethylaminoacetyloxy and 2-aminopropionyloxy;
the compound wherein R_{b} is 2,2-dimethyl-3-hydroxypropyl, 3-hydroxy-2-hydroxymethyl-2-methylpropyl, 3-acetoxy-2,2-dimethylpropyl, 3-acetoxy-2-hydroxymethyl-2-methylpropyl or 3-acetoxy-2-acetoxymethyl-2-methylpropyl;
the compound wherein R_{1b} is methyl;
the compound wherein W is chlorine atom;
the compound wherein X_{b} is a group represented by the formula:

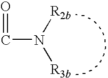

wherein, R_{2b} and R_{3b} are each a hydrogen atom, an optionally substituted hydrocarbon group, an optionally substituted heterocyclic group or an acyl group, or R_{2b} and R_{3b} may be combined together with the adjacent nitrogen atom to form an optionally substituted 5- or 6-membered nitrogen-containing heterocyclic ring optionally containing 1 to 3 hetero atoms selected from a nitrogen atom, a sulfur atom and an oxygen atom as ring constituent atoms;
the compound wherein as for a group represented by X_{b},

R_{2b} is a hydrogen atom or a C₁₋₇ alkyl group,

R_{3b} is (1) a hydrocarbon group selected from (a) C₁₋₇ alkyl, (b) C₃₋₇ cycloalkyl, (c) C₂₋₆ alkenyl, (d) C₆₋₁₀ aryl and (e) C₆₋₁₀ aryl-C₁₋₄ alkyl [wherein, (a) C₁₋₇ alkyl, (b) C₃₋₇ cycloalkyl and (c) C₂₋₆ alkenyl may be respectively substituted with 1 to 4 substituents selected from (i) carboxyl group optionally esterified with C₁₋₆ alkyl or C₆₋₁₀ aryl-C₁₋₄ alkyl, (ii) phosphate group optionally mono- or di-substituted with C₁₋₆ alkyl or C₂₋₇ alkanoyloxy-C₁₋₆ alkyl, (iii) a sulfonate group, (iv) sulfonamide group optionally substituted with C₁₋₆ alkyl or C₆₋₁₀ aryl-C₁₋₄ alkyl, (v) hydroxy group optionally alkylated with C₁₋₃ alkyl, (vi) sulfhydryl group optionally alkylated with C₁₋₃ alkyl, (vii) a carbamoyl group, (viii) phenyl group optionally substituted with 1 to 5 substituents selected from a hydroxy group, a chlorine atom, a fluorine atom, aminosulfonyl and amino group optionally mono- or di-substituted with C₁₋₃ alkyl, (ix) amino group optionally mono- or di-substituted with C₁₋₃ alkyl, (x) cyclic amino group derived from piperidine, pyrrolidine, morpholine, thiomorpholine, piperazine, 4-methylpiperazine, 4-benzylpiperazine, 4-phenylpiperazine, 1,2,3,4-tetrahydroisoquinoline or phthalimide, which may be substituted with C₁₋₃ alkyl, benzyl or phenyl and (xi) a 5- to 6-membered aromatic heterocyclic group derived from pyridine, imidazole, indole or tetrazole; and

(d) C₆₋₁₀ aryl and (e) C₆₋₁₀ aryl-C₁₋₄ alkyl may be respectively substituted with 1 to 4 substituents selected from (i) carboxyl group optionally esterified with C₁₋₄ alkyl, (ii) phosphate group optionally mono- or di-substituted with C₁₋₆ alkyl or C₂₋₇ alkanoyloxy-C₁₋₆ alkyl, (iii) a sulfonate group, (iv) a C₁₋₄ alkylsulfonyl, C₆₋₁₀ arylsulfonyl or C₆₋₁₀ aryl-C₁₋₄ alkylsulfonyl group, (v) sulfonamide group optionally substituted with C₁₋₆ alkyl or C₆₋₁₀ aryl-C₁₋₄ alkyl, (vi) C₁₋₃ alkyl group optionally substituted with carboxyl group optionally esterified with C₁₋₄ alkyl, phosphate group optionally mono- or di-substituted with C₁₋₆ alkyl, a sulfonate group, C₁₋₄ alkylsulfonyl, C₆₋₁₀ arylsulfonyl or C₆₋₁₀ aryl-C₁₋₄ alkylsulfonyl, sulfonamide group optionally substituted with C₁₋₆ alkyl or C₆₋₁₀ aryl-C₁₋₁₄ alkyl and (vii) a halogen atom],
(2) tetrazolyl, 4,5-dihydro-5-oxo-1,2,4-oxadiazolyl, 4,5-dihydro-5-thioxo-1,2,4-oxadiazolyl, 2,3-dihydro-3-oxo-1,2,4-oxadiazolyl, 2,3-dihydro-3-thioxo-1,2,4-oxadiazolyl, 3,5-dioxo-1,2,4-oxadiazolydinyl, 4,5-dihydro-5-oxo-isoxazolyl, 4,5-dihydro-5-thioxo-isoxazolyl, 2,3-dihydro-2-oxo-1,3,4-oxadiazolyl, 2,3-dihydro-3-oxo-1,2,4-tetrazolyl or 2,3-dihydro-3-thioxo-1,2,4-tetrazolyl group, or
(3) an acyl group selected from (i) a C₂₋₇ alkanoyl group which may be substituted with 1 to 2 halogen atoms, and (ii) a C₆₋₁₀ arylsulfonyl group optionally substituted with 1 to 4 substituents selected from C₁₋₃ alkyl, C₁₋₃ alkoxy and a halogen atom, a C₁₋₄ alkylsulfonyl group or a C₆₋₁₀ aryl-C₁₋₄ alkylsulfonyl group,
or R_{2b} and R_{3b} may be combined together with the adjacent nitrogen atom to form 5- or 6-membered ring derived from piperidine, piperazine, pyrrolidine, 2-oxopiperazine, 2,6-dioxopiperazine, morpholine or thiomorpholine [wherein, the 5-membered or 6-membered ring is optionally substituted with 1 to 4 substituent selected from (A) hydroxy group optionally substituted with C₁₋₃ alkyl or C₂₋₇ alkanoyl, (B) carboxyl group optionally esterified with C₁₋₆ alkyl or C₆₋₁₀ aryl-C₁₋₄ alkyl, (C) phosphate group optionally mono- or di-substituted with C₁₋₆ alkyl or C₂₋₇ alkanoyloxy-C₁₋₆ alkyl, (D) a sulfonate group, (E) sulfonamide group optionally substituted with C₁₋₆ alkyl or C₆₋₁₀ aryl-C₁₋₄ alkyl, (F) C₁₋₆ alkyl and C₂₋₅ alkenyl, each of which may be substituted with a carboxyl group optionally esterified with C₁₋₆ alkyl or C₆₋₁₀ aryl-C₁₋₄ alkyl; a phosphate group optionally mono- or di-substituted with C₁₋₆ alkyl or C₂₋₇ alkanoyloxy-C₁₋₆ alkyl; a sulfonate group; sulfonamide group optionally substituted with C₁₋₆ alkyl or C₆₋₁₀ aryl-C₁₋₄ alkyl; a hydroxy group optionally substituted with C₁₋₃ alkyl or C₂₋₇ alkanoyl; a sulfhydryl group optionally alkylated with C₁₋₃ alkyl; a carbamoyl group; phenyl optionally substituted with 1 to 5 substituents selected from a hydroxy group, a halogen atom, an aminosulfonyl and an amino group optionally substituted with C₁₋₃ alkyl; an amino group optionally mono- or di-substituted with C₁₋₃ alkyl; or tetrazolyl, (G) amino group optionally mono- or di-substituted with C₁₋₃ alkyl, (H) a cyclic amino group derived from piperidine, pyrrolidine, morpholine, thiomorpholine, 4-methylpiperazine, 4-benzylpiperazine or 4-phenylpiperazine, (I) a cyano group, (J) a carbamoyl group, (K) an oxo group, (L) a tetrazolyl or 2,5-dihydro-5-oxo-1,2,4-oxadiazolyl group, (M) carbamoyl group optionally substituted with C₆₋₁₀ arylsulfonyl, C₁₋₄ alkylsulfonyl or C₆₋₁₀ aryl-C₁₋₄ alkylsulfonyl, (N) sulfhydryl group optionally alkylated with C₁₋₃ alkyl, and (O) phenyl group which may be substituted with 1 to 5 substituents selected from a hydroxy group, a halogen atom, aminosulfonyl and amino group optionally substituted with C₁₋₃ alkyl];
the compound wherein in a group represented by X_{b}, R_{2b} and R_{3b} may be combined together with the adjacent nitrogen atom of carbamoyl group to form a 5- or 6-membered ring derived from piperidine, piperazine, pyrrolidine, 2-oxopiperazine or 2,6-dioxopiperazine, and the 5- or 6-membered ring may be substituted with C₁₋₆ alkyl group optionally having 1 to 2 substituents selected from (i) carboxyl group optionally esterified with C₁₋₆ alkyl or C₆₋₁₀ aryl-C₁₋₄ alkyl, (ii) phosphate group optionally mono- or di-substituted with C₁₋₆ alkyl or C₂₋₇ alkanoyl-C₁₋₆ alkyl, (iii) a sulfonate group, (iv) sulfonamide group optionally substituted with C₁₋₆ alkyl or C₆₋₁₀ aryl-C₁₋₄ alkyl, (v) hydroxy group optionally alkylated with C₁₋₃ alkyl, (vi) sulfhydryl group optionally alkylated with C₁₋₃ alkyl, (vii) a carbamoyl group, (viii) phenyl group which may be substituted with 1 to 5 substituents selected from a hydroxy group, a halogen atom, aminosulfonyl and amino optionally substituted with C₁₋₃ alkyl, (ix) amino group optionally mono- or di-substituted with C₁₋₃ alkyl, and (x) a tetrazolyl group;
the compound wherein in a group represented by X_{b}, R_{2b} is a hydrogen atom or C₁₋₇ alkyl and R_{3b} is C₁₋₄ alkylsulfonyl;
the compound wherein the heterocyclic group represented by X_{b} is tetrazolyl, 4,5-dihydro-5-oxo-1,2,4-oxadiazolyl, 4,5-dihydro-5-thioxo-1,2,4-oxadiazolyl, 2,3-dihydro-3-oxo-1,2,4-oxadiazolyl, 2,3-dihydro-3-thioxo-1,2,4-oxadiazolyl, 3,5-dioxo-1,2,4-oxadiazolydinyl, 4,5-dihydro-5-oxo-isoxazolyl, 4,5-dihydro-5-thioxo-isoxazolyl, 2,3-dihydro-2-oxo-1,3,4-oxadiazolyl, 2,3-dihydro-3-oxo-1,2,4-tetrazolyl or 2,3-dihydro-3-thioxo-1,2,4-tetrazolyl;
the compound wherein R_{1b} is methyl, W is a chlorine atom, R_{b} is C₃₋₆ branched alkyl which is substituted with 1 to 3 substituents selected from a hydroxy group, acetyloxy, propionyloxy, tert-butoxycarbonyloxy, palmitoyloxy, dimethylaminoacetyloxy and 2-aminopropionyloxy, and X_{b} is a group represented by the formula:

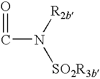

wherein, R_{2b′} represents a hydrogen atom or C₁₋₇ alkyl and R_{3b′} represents C₁₋₄ alkyl;
the compound wherein R_{1b} is methyl, W is a chlorine atom, R_{b} is C₃₋₆ branched alkyl which is substituted with 1 to 3 substituents selected from a hydroxy group, acetyloxy, propionyloxy, tert-butoxycarbonyloxy, palmitoyloxy, dimethylaminoacetyloxy and 2-aminopropionyloxy, and X_{b} is a group represented by the formula:

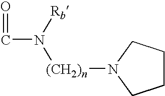

wherein, R_{b′} represents a hydrogen atom or C₁₋₇ alkyl, and n represents an integer of 1 to 5;
the compound wherein R_{1b} is methyl, W is a chlorine atom, R_{b} is C₃₋₆ branched alkyl which is substituted with 1 to 3 substituents selected from a hydroxy group, acetyloxy, propionyloxy, tert-butoxycarbonyloxy, palmitoyloxy, dimethylaminoacetyloxy and 2-aminopropionyloxy, and X_{b} is a group represented by the formula:

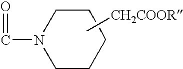

wherein, R″ represents a hydrogen atom or C₁₋₄ alkyl;
the compound wherein R_{1b} is methyl, W is a chlorine atom, R_{b} is C₃₋₆ branched alkyl which is substituted with 1 to 3 substituents selected from a hydroxy group, acetyloxy, propionyloxy, tert-butoxycarbonyloxy, palmitoyloxy, dimethylaminoacetyloxy and 2-aminopropionyloxy, and X_{b} is tetrazolyl;
the compound wherein R_{b} is lower alkyl optionally substituted with 1 or 2 hydroxy groups, and X_{b} is (1) carbamoyl group optionally substituted with a hydrocarbon group selected from (a) C₁₋₇ alkyl, (b) C₃₋₇ cycloalkyl, (c) C₂₋₆ alkenyl, (d) C₆₋₁₀ aryl and (e) C₇₋₁₄ arylalkyl [wherein, (a) C₁₋₇ alkyl, (b) C₃₋₇ cycloalkyl and (c) C₂₋₆ alkenyl may respectively have 1 to 4 substituents selected from (i) carboxyl group optionally esterified with C₁₋₆ alkyl or C₇₋₁₀ arylalkyl, (ii) a phosphate group, (iii) a sulfonate group, (iv) sulfonamide group optionally substituted with C₁₋₆ alkyl or C₇₋₁₀ arylalkyl, (v) hydroxy group optionally alkylated with C₁₋₃ alkyl, (vi) sulfhydryl group optionally alkylated with C₁₋₃ alkyl, (vii) a carbamoyl group, (viii) an phenyl group optionally substituted with substituents selected from a hydroxy group, a chlorine atom, a fluorine atom, aminosulfonyl and amino group optionally mono- or di-substituted with C₁₋₃ alkyl, (ix) amino group optionally mono- or di-substituted with C₁₋₃ alkyl, and (x) cyclic amino group derived from piperidine, pyrrolidine, morpholine, thiomorpholine, piperazine, 4-methylpiperazine, 4-benzylpiperazine, or 4-phenylpiperazine, which may be substituted with C₁₋₃ alkyl, benzyl or phenyl and (xi) 5- or 6-membered aromatic heterocyclic group derived from pyridine, imidazole, indole or tetrazole, and (d) C₆₋₁₀ aryl and (e) C₇₋₁₄ arylalkyl may respectively have 1 to 4 substituents selected from (i) carboxyl group optionally esterified with C₁₋₄ alkyl, (ii) a phosphate group, (iii) a sulfonate group, (iv) sulfonamide group optionally substituted with C₁₋₆ alkyl or C₇₋₁₀ arylalkyl, (v) C₁₋₃ alkyl group optionally substituted with carboxyl group which may be esterified with C₁₋₄ alkyl, a phosphate group, a sulfonate group, or sulfonamide group optionally substituted with C₁₋₆ alkyl or C₇₋₁₀ arylalkyl, and (iv) a halogen atom],
(2) tetrazolyl, 4,5-dihydro-5-oxo-1,2,4-oxadiazolyl, 4,5-dihydro-5-thioxo-1,2,4-oxadiazolyl, 2,3-dihydro-3-oxo-1,2,4-oxadiazolyl, 2,3-dihydro-3-thioxo-1,2,4-oxadiazolyl, 3,5-dioxo-1,2,4-oxadiazolydinyl, 4,5-dihydro-5-oxo-isoxazolyl, 4,5-dihydro-5-thioxo-isoxazolyl, 2,3-dihydro-2-oxo-1,3,4-oxadiazolyl, 2,3-dihydro-3-oxo-1,2,4-tetrazolyl or 2,3-dihydro-3-thioxo-1,2,4-tetrazolyl group,
(3) carbamoyl group optionally substituted with an acyl group selected from (i) a C₂₋₇ alkanoyl group optionally substituted with 1 to 2 halogen atoms, and
(ii) a C₆₋₁₀ arylsulfonyl group, a C₁₋₄ alkylsulfonyl group or a C₇₋₁₄ arylalkylsulfonyl group, each of which may be substituted with 1 to 4 substituents selected from C₁₋₃ alkyl, C₁₋₃ alkoxy and a halogen atom, or
(4) a cyclic amino carbonyl group derived from piperidine, piperazine, pyrrolidine, 2-oxopiperazine, 2,6-dioxopiperazine, morpholine and thiomorpholine [wherein, the cyclic amino carbonyl group may have 1 to 4 substituents selected from (A) a hydroxy group, (B) carboxyl group optionally esterified with C₁₋₄ alkyl, (C) a phosphate group, (D) a sulfonate group, (E) sulfonamide group optionally substituted with C₁₋₆ alkyl or C₇₋₁₀ arylalkyl, (F) C₁₋₃ alkyl or C₂₋₅ alkenyl, each of which may be substituted with the above-mentioned (A), (B), (C), (D) or (E), (G) amino group optionally mono- or di-substituted with C₁₋₃ alkyl, (H) a cyclic amino group derived from piperidine, pyrrolidine, morpholine, thiomorpholine, 4-methylpiperazine, 4-benzylpiperazine or 4-phenylpiperazine, (I) a cyano group, (J) a carbamoyl group, (K) oxo, (L) C₁₋₃ alkoxy, (M) a heterocyclic group derived from tetrazolyl or 2,5-dihydro-5-oxo-1,2,4-oxadiazolyl, and (N) carbamoyl group optionally substituted with C₆₋₁₀ arylsulfonyl, C₁₋₄ alkylsulfonyl or C₇₋₁₄ arylalkylsulfonyl];
the compound wherein R_{b} is a 2,2-dimethyl-3-hydroxypropyl group; or the like.

In the aforementioned formula, examples of the lower alkyl group represented by R_{b} include C₁₋₆ alkyl such as methyl, ethyl, n-propyl, isopropyl, n-butyl, isobutyl, n-pentyl, isopentyl, neopentyl and hexyl. Among these, a C₃₋₆ alkyl group is preferred, and a C₄₋₅ alkyl group is more preferred. In particular, a branched C₄₋₅ alkyl group such as isobutyl and neopentyl is preferred.

Examples of the substituent of the lower alkyl represented by R_{b} include hydroxy group optionally substituted with C₂₋₂₀ alkanoyl or C₁₋₇ alkyl, and the like. Examples of these substituents include a hydroxy group, acetyloxy, propionyloxy, tert-butoxycarbonyloxy, palmitoyloxy, dimethylaminoacetyloxy and 2-aminopropionyloxy.

One to three of these substituents may be present at their substitutable positions.

In addition, examples of the optionally substituted lower alkyl represented by R_{b} include 2,2-dimethyl-3-hydroxypropyl, 3-hydroxy-2-hydroxymethyl-2-methylpropyl, 3-acetoxy-2,2-dimethylpropyl, 3-acetoxy-2-hydroxymethyl-2-methyl-propyl and 3-acetoxy-2-acetoxymethyl-2-methylpropyl.

The optionally substituted carbamoyl group represented by X_{b} includes a group represented by the formula:

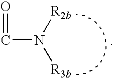

Examples of the “optionally substituted hydrocarbon group” represented by R_{2b} and R_{3b} include an optionally substituted C₁₋₇ linear or branched alkyl group (e.g., methyl, ethyl, n-propyl, isopropyl, n-butyl, isobutyl, 1,1-dimethylethyl, n-pentyl, 3-methylbutyl, 2-methylbutyl, 1-methylbutyl, 1-ethylpropyl, n-hexyl, 4-methylpentyl, 3-methylpentyl, 2-methylpentyl, 2-ethylbutyl, 1-ethylbutyl, neopentyl, hexyl, heptyl), an optionally substituted C₃₋₇ cycloalkyl group (e.g., cyclopropyl, cyclobutyl, cyclopentyl, cyclohexyl, cyclohexylmethyl, etc.), an optionally substituted C₂₋₆ linear or branched alkenyl group (e.g., vinyl, allyl, isopropenyl, 2-methylallyl, 1-propenyl, 2-methyl-1-propenyl, 2-methyl-2-propenyl, 1-butenyl, 2-butenyl, 3-butenyl, 2-ethyl-1-butenyl, 2-methyl-2-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl, 5-hexenyl etc.), an optionally substituted C₆₋₁₀ aryl group (e.g., phenyl and naphthyl group) and an optionally substituted C₇₋₁₄ arylalkyl group (e.g., benzyl, phenethyl and naphthylmethyl).

Examples of the substituent of the “optionally substituted C₁₋₇ linear or branched alkyl group, optionally substituted C₃₋₇ cycloalkyl group and optionally substituted C₂₋₆ linear or branched alkenyl group” include carboxyl group optionally esterified with C₁₋₆ alkyl group or C₆₋₁₀ aryl-C₁₋₄ alkyl group (e.g., methyl, ethyl, propyl, isopropyl, butyl, tert-butyl, phenyl, benzyl, etc.), phosphate group optionally mono- or di-substituted with C₁₋₆ alkyl group (e.g., methyl, ethyl, n-propyl, isopropyl, n-butyl, isobutyl, n-pentyl, isopentyl, neopentyl, hexyl, etc.) or C₂₋₇ alkanoyloxy-C₁₋₆ alkyl such as acetyloxymethyl or pivaloyloxymethyl group, a sulfonate group, sulfonamide group optionally substituted with C₁₋₆ alkyl group or C₆₋₁₀ aryl-C₁₋₄ alkyl group (e.g., methyl, ethyl, propyl, isopropyl, butyl, tert-butyl, benzyl, etc.), hydroxy group and sulfhydryl group, each optionally alkylated with C₁₋₃ alkyl group (e.g., methyl, ethyl, propyl, etc.), a carbamoyl group, phenyl group optionally substituted with 1 to 5 substituents [e.g., a hydroxy group, chlorine, fluorine, an aminosulfonyl group or amino group optionally substituted with C₁₋₃ alkyl group (e.g., methyl, ethyl, propyl, etc.)], an amino group optionally mono- or di-substituted with C₁₋₃ alkyl group (e.g., methyl, ethyl, propyl, etc.), a cyclic amino group (e.g., 5- or 6-membered cyclic amino group derived from cyclic amine such as piperidine, pyrrolidine, morpholine, thiomorpholine, piperazine, 4-methylpiperazine, 4-benzylpiperazine, 4-phenylpiperazine, 1,2,3,4-tetrahydroisoquinoline and phthalimide, which may be substituted with a C₁₋₃ alkyl group, benzyl, phenyl or the like, and further optionally contains an oxygen atom or a sulfur atom as a ring constituent atom) and a 5- to 6-membered aromatic heterocyclic ring containing 1 to 4 hetero atoms selected from N, O and S (e.g., pyridine, imidazole, indole, tetrazole, etc).

In addition, examples of the substituent of C₆₋₁₀ aryl group and C₆₋₁₀ aryl-C₁₋₁₄ alkyl group as the substituent of an optionally substituted amino group which composes a carbamoyl group of the “optionally substituted carbamoyl group” represented by X_{b} include carboxyl group optionally esterified with C₁₋₄ alkyl group (methyl, ethyl, propyl, tert-butyl group, etc.), phosphate group optionally mono- or di-substituted with C₁₋₆ alkyl group (methyl, ethyl, n-propyl, isopropyl, n-butyl, isobutyl, n-pentyl, isopentyl, neopentyl, hexyl) or C₂₋₇ alkanoyloxy-C₁₋₆ alkyl such as pivaloyloxymethyl group and acetyloxymethyl group, a sulfonate group, C₁₋₄ alkylsulfonyl, C₆₋₁₀ arylsulfonyl or C₆₋₁₀ aryl-C₁₋₄ alkylsulfonyl, sulfonamide group optionally substituted with a C₁₋₆ alkyl group or a C₆₋₁₀ aryl-C₁₋₄ alkyl group (e.g., methyl, ethyl, propyl, isopropyl, butyl, tert-butyl, benzyl, etc.) and carboxyl group optionally esterified with a C₁₋₄ alkyl group, phosphate group optionally mono- or di-substituted with a C₁₋₆ alkyl group such as methyl, ethyl, n-propyl, isopropyl, n-butyl, isobutyl, n-pentyl, isopentyl, neopentyl and hexyl or C₂₋₇ alkanoyloxy-C₁₋₆ alkyl group such as pivaloyloxymethyl group, C₁₋₃ alkyl group (e.g., methyl, ethyl, propyl and isopropyl) optionally substituted with a sulfonate group and sulfonamide group optionally substituted with C₁₋₆ alkyl or a C₆₋₁₀ aryl-C₁₋₄ alkyl group, and a halogen atom (e.g., fluorine and chlorine), and the like.

The “hydrocarbon group” may have 1 to 5 substituents at a substitutable position.

The “optionally substituted heterocyclic group” represented by R_{2b} and R_{3b} may have 1 to 2 (preferably one) of substituents such as oxo group and thioxo group, and preferred is a heterocyclic group having a hydrogen atom which can be deprotonated. Such heterocyclic group is preferably a 5- to 6-membered heterocyclic group containing 1 to 4, preferably 2 to 3 hetero atoms selected from S, O and N. Specific examples include tetrazolyl, 4,5-dihydro-5-oxo-1,2,4-oxadiazolyl, 4,5-dihydro-5-thioxo-1,2,4-oxadiazolyl, 2,3-dihydro-3-oxo-1,2,4-oxadiazolyl, 2,3-dihydro-3-thioxo-1,2,4-oxadiazolyl, 3,5-dioxo-1,2,4-oxadiazolydinyl, 4,5-dihydro-5-oxo-isoxazolyl, 4,5-dihydro-5-thioxo-isoxazolyl, 2,3-dihydro-2-oxo-1,3,4-oxadiazolyl, 2,3-dihydro-3-oxo-1,2,4-tetrazolyl and 2,3-dihydro-3-thioxo-1,2,4-tetrazolyl. Among these, tetrazolyl group is preferred.

Examples of the “acyl group” represented by R_{2b} and R_{3b} include a carboxylic acid acyl group derived from carboxylic acid (e.g., C₂₋₇ carboxylic acid acyl group such as acetyl, propionyl, butyryl, benzoyl, etc.) and C₆₋₁₀ arylsulfonyl group, C₁₋₄ alkylsulfonyl group and C₆₋₁₀ aryl-C₁₋₄ alkylsulfonyl group, each of which may have a substituent (e.g., methylsulfonyl, ethylsulfonyl, phenylsulfonyl, naphthylsulfonyl, phenylmethylsulfonyl, phenylethylsulfonyl, naphthylmethylsulfonyl, naphthylethylsulfonyl, etc.). The substituents of aryl, alkyl and arylalkylsulfonyl group include C₁₋₃ alkyl (e.g., methyl, ethyl, propyl, etc.), C₁₋₃ alkoxy (e.g., methoxy, ethoxy, propoxy, etc.), a halogen atom (e.g., chlorine, fluorine, bromine) and the like, and 1 to 4, preferably 1 to 2, thereof may be present at a substitutable position.

The aforementioned carboxylic acid acyl group may have 1 to 2 halogen atoms (e.g., chlorine, fluorine, bromine) as a substituent.

Examples of the cyclic amino group optionally substituted with C₁₋₃ alkyl, C₂₋₇ alkanoyl or the like, which is formed by combining R_{2b} and R_{3b} together with the adjacent nitrogen atom of carbamoyl group, include a group derived from 5- or 6-membered cyclic amine such as piperazine, piperidine, pyrrolidine, piperazin-2-one, piperazine-2,6-dione, morpholine and thiomorpholine, and said cyclic amine may further contain 1 to 3 hetero atoms selected from a nitrogen atom, a sulfur atom and an oxygen atom as ring constituent atom. Such cyclic amino group may have 1 to 4, preferably 1 to 2 substituents. Examples of the substituents include hydroxy group optionally substituted with C₁₋₃ alkyl group or C₂₋₇ alkanoyl, carboxyl group optionally esterified with C₁₋₄ alkyl group (methyl, ethyl, propyl, tert-butyl, etc.) and C₇₋₁₀ arylalkyl, phosphate group optionally mono- or di-substituted with C₁₋₆ alkyl or C₂₋₇ alkanoyloxy-C₁₋₆ alkyl group (acetyloxymethyl group, pivaloyloxymethyl group), a sulfonate group, and sulfonamide group optionally substituted with C₁₋₆ alkyl or C₆₋₁₀ aryl-C₁₋₄ alkyl group (e.g., methyl, ethyl, propyl, isopropyl, butyl, tert-butyl, benzyl, etc.), C₁₋₆ alkyl and C₂₋₅ alkenyl, each of which may be substituted with “carboxyl group optionally esterified with C₁₋₆ alkyl or C₆₋₁₀ aryl-C₁₋₄ alkyl, phosphate group optionally mono- or di-substituted with C₁₋₆ alkyl or C₂₋₇ alkanoyloxy-C₁₋₆ alkyl group (e.g., acetyloxymethyl group, pivaloyloxymethyl group, etc.), a sulfonate group, sulfonamide group optionally substituted with C₁₋₆ alkyl or C₆₋₁₀ aryl-C₁₋₄ alkyl group, hydroxy group optionally substituted with C₁₋₃ alkyl or C₂₋₇ alkanoyl, sulfhydryl group optionally alkylated with C₁₋₃ alkyl, a carbamoyl group, phenyl group optionally substituted with 1 to 5 substituents (e.g., a hydroxy group, a halogen atom, aminosulfonyl, amino group optionally substituted with C₁₋₃ alkyl, etc.), amino group optionally mono- or di-substituted with C₁₋₃ alkyl or tetrazolyl group”, amino group optionally mono- or di-substituted with C₁₋₃ alkyl (e.g., methyl, ethyl, propyl, etc.), a cyclic amino group (a group derived from 5- or 6-membered cyclic amine which may contain additional hetero atoms selected from a nitrogen atom, a sulfur atom and an oxygen as ring constituent atom, and which may be substituted with C₁₋₃ alkyl, benzyl, phenyl, or the like, for example, piperidine, pyrrolidine, morpholine, thiomorpholine, 4-methylpiperazine, 4-benzylpiperazine, 4-phenylpiperazine, or the like), a cyano group, a carbamoyl group, an oxo group, C₁₋₃ alkoxy (e.g., methoxy, ethoxy, ethylenedioxy, etc.), heterocyclic group optionally substituted with an oxo group or thioxo group having a hydrogen atom which can be deprotonated as mentioned above (e.g., tetrazolyl, 2,5-dihydro-5-oxo-1,2,4-oxadiazolyl, etc.), C₆₋₁₀ arylsulfonyl, C₆₋₁₀ aryl-C₁₋₄ alkylsulfonyl and C₁₋₄ alkylsulfonyl (e.g., methylsulfonyl, ethylsulfonyl, propylsulfonyl, butylsulfonyl, isopropylsulfonyl, tert-butylsulfonyl, phenylsulfonyl, benzylsulfonyl, etc.) which is exemplified for the substituent of an optionally substituted amino group composing carbamoyl of the “optionally substituted carbamoyl group” represented by X, sulfhydryl group optionally alkylated with C₁₋₃ alkyl, or carbamoyl group substituted with phenyl which may be substituted with 1 to 5 substituents (e.g., a hydroxy group, a halogen atom, an aminosulfonyl and amino group optionally substituted with C₁₋₃ alkyl).

Examples of the optionally substituted carbamoyl group represented by X_{b} include:

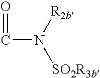

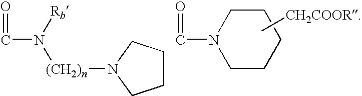

R_{2b′} and R_{b′} include a hydrogen atom and C₁₋₇ alkyl. Hydrogen atom is particularly preferred.

The C₁₋₇ alkyl represented by R_{2b}, R_{2b′} and R_{b′} includes the same groups as those exemplified with respect to the aforementioned C₁₋₇ alkyl of the “hydrocarbon group”.

R″ includes a hydrogen atom and C₁₋₄ alkyl. Hydrogen atom is particularly preferred.

The C₁₋₄ alkyl represented by R_{3b′} and R″ includes, for example, methyl, ethyl, propyl, isopropyl, n-butyl, tert-butyl, etc.

As for the optionally substituted heterocyclic group having a hydrogen atom which can be deprotonated represented by X_{b}, a nitrogen-containing (preferably containing 1 to 4 nitrogen atoms) 5- to 6-membered heterocyclic ring having Broensted acid-like active proton is preferred, and those containing 1 to 4, preferably 2 to 3 of a nitrogen atom, a sulfur atom and an oxygen atom may be preferred. The substituents thereof include an oxo group and a thioxo group, and 1 to 2, preferably 1, of such substituents may be present. Examples of the “optionally substituted heterocyclic group having a hydrogen atom which can be deprotonated” represented by X are exemplified by those for the “optionally substituted heterocyclic group” as the substituent of the “optionally substituted carbamoyl group” represented by X such as tetrazolyl, 2,5-dihydro-5-oxo-1,2,4-oxadiazolyl and the like.

Examples of the “lower alkyl group” represented by R_{1b} include a C₁₋₆ alkyl group such as methyl, ethyl, n-propyl, isopropyl, n-butyl, tert-butyl, pentyl, hexyl and the like. Among these, C₁₋₃ alkyl group is preferred. In the view of pharmacological activity, methyl group is particularly preferred as R_{1b}.

Examples of the “halogen atom” represented by W include chlorine, fluorine, bromine, iodine atoms. The chlorine atom is particularly preferred.

Examples of the salts of the compound represented by the formula (Ib) include pharmacologically acceptable salts such as inorganic salts such as hydrochloride, hydrobromide, sulfate, nitrate, phosphate and the like; organic salts such as acetate, tartrate, citrate, fumarate, maleate, toluenesulfonate, methanesulfonate and the like; metal salts such as sodium salt, potassium salt, calcium salt, aluminum salt and the like; and salts with base such as triethylamine salt, guanidine salt, ammonium salt, hydrazine salt, quinine salt, cinchonine salt and the like.

In addition, hydrates as well as non-hydrates of the compound represented by the formula (Ib) are included within the scope of the present invention.

The compound represented by the formula (Ib) and salts thereof contains asymmetric carbon atoms at 3- and 5-positions, herein the trans isomer wherein the substituents on 3- and 5-positions are directed in the opposite direction relative to the plane of a 7-membered ring is preferred, and in particular, the isomer wherein the absolute configuration at 3-position is R-configuration and the absolute configuration at 5-position is S-configuration is preferred.

As for the compounds represented by the formula (Ib) or salts thereof, the following compounds are specifically preferred.
N-methanesulfonyl-[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetamide,
N-methanesulfonyl-[(3R,5S)-7-chloro-5-(2,3-dimethoxyphenyl)-1-(3-hydroxy-2-hydroxymethyl-2-methylpropyl)-2-oxo-1,2,3,5-tetrahydro-4,1-benzoxazepin-3-yl]acetamide,
N-[2-(pyrrolidin-1-yl)ethyl]-[(3R,5S)-7-chloro-5-(2,3-dimethoxyphenyl)-1-(3-hydroxy-2-hydroxymethyl-2-methylpropyl)-2-oxo-1,2,3,5-tetrahydro-4,1-benzoxazepin-3-yl]acetamide,
N-[2-(pyrrolidin-1-yl)ethyl]-[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetamide,
N-methanesulfonyl-[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetamide,
N-methanesulfonyl-[(3R,5S)-1-(3-acetoxy-2-acetoxymethyl-2-methylpropyl)-7-chloro-5-(2,3-dimethoxyphenyl)-2-oxo-1,2,3,5-tetrahydro-4,1-benzoxazepin-3-yl]acetamide,
N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidin-4-acetic acid,
N-[[(3R,5S)-1-(3-hydroxy-2,2-dimethylpropyl)-7-chloro-5-(2,3-dimethoxyphenyl)-2-oxo-1,2,3,5-tetrahydro-4,1-benzoxazepin-3-yl]acetyl]piperidin-4-acetic acid,
N-[[(3R,5S)-1-(2,2-dimethylpropyl)-7-chloro-5-(2,3-dimethoxyphenyl)-2-oxo-1,2,3,5-tetrahydro-4,1-benzoxazepin-3-yl]acetyl]piperidin-4-acetic acid,
N-[[(3R,5S)-1-(3-acetoxy-2-acetoxymethyl-2-methylpropyl)-7-chloro-5-(2,3-dimethoxyphenyl)-2-oxo-1,2,3,5-tetrahydro-4,1-benzoxazepin-3-yl]acetyl]piperidin-4-acetic acid,
ethyl N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetate,
ethyl N-[[(3R,5S)-1-(3-acetoxy-2-acetoxymethyl-2-methylpropyl)-7-chloro-5-(2,3-dimethoxyphenyl)-2-oxo-1,2,3,5-tetrahydro-4,1-benzoxazepin-3-yl]acetyl]piperidine-4-acetate,
(3R,5S)-7-chloro-5-(2,3-dimethoxyphenyl)-1-(3-hydroxy-2,2-dimethylpropyl)-1,2,3,5-tetrahydro-3-[1H (or 3H)-tetrazol-5-yl]methyl-4,1-benzoxapin-2-one,
(3R,5S)-7-chloro-5-(2,3-dimethoxyphenyl)-1-(3-hydroxy-2-hydroxymethyl-2-methylpropyl)-1,2,3,5-tetrahydro-3-[1H (or 3H)-tetrazol-5-yl]methyl-4,1-benzoxazepin-2-one,
(3R,5S)-1-(3-acetoxy-2,2-dimethylpropyl)-7-chloro-5-(2,3-dimethoxyphenyl)-1,2,3,5-tetrahydro-3-[1H (or 3H)-tetrazol-5-yl]methyl-4,1-benzoxazepin-2-one,
(3R,5S)-1-(3-acetoxy-2-acetoxymethyl-2-methylpropyl)-7-chloro-5-(2,3-dimethoxyphenyl)-1,2,3,5-tetrahydro-3-[1H (or 3H)-tetrazol-5-yl]methyl-4,1-benzoxazepin-2-one,
N-[2-(pyrrolidin-1-yl)ethyl]-[(3R,5S)-7-chloro-5-(2,3-dimethoxyphenyl)-1-neopentyl-2-oxo-1,2,3,5-tetrahydro-4,1-benzoxazepin-3-yl]acetamide, and the like.

The compound represented by the formula (Ib) and salts thereof can be prepared according to the methods disclosed in the publications, for example, EP-A-567026, WO95/21834 (PCT application based on Japanese Patent Application No. 6-15531), EP-A-645377 (an application based on Japanese Patent Application No. 6-229159), EP-A-645378 (an application based on Japanese Patent Application No. 6-229160), WO97/10224 and the like, or the methods similar thereto.

As the compound represented by the formula (I), the compound represented by the aforementioned formula (Ic):

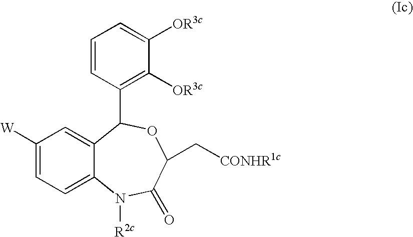

is preferred.

Preferable examples of the compound represented by the formula (Ic) include:

the compound wherein R^{1c} is a 3-carboxypropyl group, a 1-carboxyethyl group, or a C₃₋₆ linear alkyl-sulfonyl group, a (carboxy-C₅₋₇ cycloalkyl)-C₁₋₃ alkyl group, a (carboxyfuryl)-alkyl group, a carboxy-C₆₋₁₀ aryl group, a (carboxy-C₂₋₃ alkyl)-C₆₋₁₀ aryl group or a (carboxy-C₁₋₃ alkyl)-C₇₋₁₄ aralkyl group, each of which may be optionally substituted;
the compound wherein R^{1c} is a (carboxy-C₁₋₄ alkyl)-C₆₋₁₀ aryl group which may have a substituent;
the compound wherein R^{1c} is a (carboxy-C₂₋₃ alkyl)-C₆₋₁₀ aryl group which may have a substituent;
the compound wherein R^{1c} is a (carboxy-C₂₋₃ alkyl)-phenyl group which may have a substituent;
the compound wherein R^{1c} is a (carboxyfuryl)-alkyl group which may have a substituent;
the compound wherein R^{2c} is a C₃₋₆ alkyl group which have alkanoyloxy group and/or a hydroxy group;
the compound wherein R^{2c} is a C₃₋₆ alkyl group which may have 1 to 3 substituents selected from hydroxy group, acetoxy, propionyloxy, tert-butoxycarbonyloxy and palmitoyloxy;
the compound wherein R^{2c} is 2,2-dimethylpropyl, 3-hydroxy-2,2-dimethylpropyl or 3-acetoxy-2,2-dimethylpropyl;
the compound wherein R^{3c} is methyl group;
the compound wherein W is chlorine atom;
the compound having R-configuration at 3-position, and S-configuration at 5-position; and the like.

In the aforementioned formula, R^{1c} represents an optionally substituted 1-carboxyethyl group, an optionally substituted carboxy-C₃₋₆ linear alkyl group, an optionally substituted C₃₋₆ linear alkyl-sulfonyl group, an optionally substituted (carboxy-C₅₋₇ cycloalkyl)-C₁₋₃ alkyl group, or a group represented by formula —X^{1c}—X^{2c}—Ar—X^{3c}—X^{4c}—COOH (wherein X^{1c} and X^{4c} respectively represents a bond or an optionally substituted C₁₋₄ alkylene group, X^{2c} and X^{3c} respectively represents a bond, —O— or —S—, and Ar represents an optionally substituted divalent aromatic ring group. Provided that, when X^{1c} is a bond, X^{2c} represents a bond, and when X^{4c} is a bond, X^{3c} represents a bond).

Examples of the C₃₋₆ linear alkyl group in the optionally substituted carboxy-C₃₋₆ linear alkyl group represented by R^{1c} include n-propyl, n-butyl, n-pentyl, n-hexyl. Among these, n-propyl and n-butyl are preferred, and n-propyl is more preferred.

Examples of the C₃₋₆ linear alkyl group in the optionally substituted C₃₋₆ linear alkyl-sulfonyl group represented by R^{1c} include n-propyl, n-butyl, n-pentyl, n-hexyl. Among these, n-propyl and n-butyl are preferred, and n-propyl is more preferred.

Examples of the C₅₋₇ cycloalkyl group in the optionally substituted (carboxy-C₅₋₇ cycloalkyl)-C₁₋₃ alkyl group represented by R^{1c} include cyclopentyl, cyclohexyl and cycloheptyl. Among these, cyclopentyl and cyclohexyl are preferred, and cyclohexyl is more preferred.

Examples of the C₁₋₃ alkyl group in the optionally substituted (carboxy-C₅₋₇ cycloalkyl)-C₁₋₃ alkyl group represented by R^{1c} include methyl, ethyl, n-propyl and isopropyl. Among these, methyl and ethyl are preferred, and methyl is more preferred.

In the group represented by formula —X^{1c}—X^{2c}—Ar—X^{3c}—X^{4c}—COOH for R^{1c}, examples of the “C₁₋₄ alkylene group” in the “optionally substituted C₁₋₄ alkylene group” represented by X^{1c} and X^{4c} include methylene, dimethylene, trimethylene, tetramethylene, and C₁₋₃ alkylene group is preferred, and among them, the linear one may be preferably used.

Examples of the “divalent aromatic ring group” in the “optionally substituted divalent aromatic ring group” represented by Ar include a divalent aromatic hydrocarbon group, a divalent aromatic heterocyclic group, and the like.

Herein, examples of the divalent aromatic hydrocarbon group include a group formed by removing one hydrogen atom from C₆₋₁₀ aryl group (e.g., phenyl, naphthyl, etc.), and phenylene is preferably used as a divalent aromatic hydrocarbon group.

Examples of the divalent aromatic heterocyclic group include a group formed by removing one hydrogen atom from an aromatic heterocyclic group containing at least 1 (preferably 1 to 4, more preferably 1 to 2) of 1 to 3 (preferably 1 to 2) kinds of hetero atoms selected from an oxygen atom, a sulfur atom and a nitrogen atom as ring-system constituent atoms (ring atom).

Herein, examples of the aromatic heterocyclic group include a 5- to 6-membered aromatic monocyclic heterocyclic group such as furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyridazinyl, pyrimidinyl, pyrazinyl and triazinyl (preferably, furyl, thienyl, pyrrolyl, imidazolyl, thiazolyl, pyridyl, etc.) and an 8- to 12-membered aromatic fused heterocyclic group such as benzofuranyl, isobenzofuranyl, benzo[b]thienyl, indolyl, isoindolyl, 1H-indazolyl, benzimidazolyl, benzoxazolyl, 1,2-benzoisoxazolyl, benzothiazolyl, benzopyranyl, 1,2-benzoisothiazolyl, 1H-benzotriazolyl, quinolyl, isoquinolyl, cinnolyl, quinazolinyl, quinoxalinyl, phthalazinyl, naphthyridinyl, purinyl, pteridinyl, carbazolyl, α-carbolinyl, β-carbolinyl, γ-carbolinyl, acridinyl, phenoxazinyl, phenothiazinyl, phenazinyl, phenoxathiinyl, thianthrenyl, phenathridinyl, phenanthrolinyl, indolizinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl and 1,2,4-triazolo[4,3-b]pyridazinyl (preferably, a heterocyclic group in which the aforementioned 5- to 6-membered aromatic monocyclic heterocyclic group is fused with a benzene ring, or a heterocyclic group in which two of the same or different 5- to 6-membered aromatic monocyclic heterocyclic groups mentioned above are fused, and more preferably, a heterocyclic group in which the aforementioned 5- to 6-membered aromatic monocyclic heterocyclic group is fused with a benzene ring).

Examples of the substituent which may be possessed by the “C₁₋₄ alkylene group” in the “optionally substituted C₁₋₄ alkylene group” represented by X^{1c} and X^{4c}; and the “divalent aromatic ring group” in the “optionally substituted divalent aromatic ring group” include: (i) carboxyl group optionally esterified with a C₁₋₆ alkyl group or a C₆₋₁₀ aryl-C₁₋₄ alkyl group (e.g., methyl, ethyl, propyl, isopropyl, butyl, tert-butyl, phenyl, benzyl, etc.), (ii) phosphate group optionally mono- or di-substituted with C₁₋₆ alkyl (e.g., methyl, ethyl, n-propyl, isopropyl, n-butyl, isobutyl, n-pentyl, isopentyl, neopentyl, hexyl, etc.) or C₂₋₇ alkanoyloxy-C₁₋₆ alkyl such as acetoxymethyl and pivaloyloxymethyl group, (iii) a sulfonate group, (iv) sulfonamide group optionally substituted with a C₁₋₆ alkyl group or a C₆₋₁₀ aryl-C₁₋₄ alkyl group (e.g., methyl, ethyl, propyl, isopropyl, butyl, tert-butyl, benzyl, etc.), (v) hydroxy group and a sulfhydryl group, each of which may be alkylated with a C₁₋₃ alkyl group (e.g., methyl, ethyl, propyl, etc.), (vi) a carbamoyl group, (vii) phenyl group which may be substituted with 1 to 5 substituents [e.g. a hydroxy group, chlorine, fluorine, aminosulfonyl group, and amino group optionally substituted with C₁₋₃ alkyl group (e.g. methyl, ethyl, propyl, etc.)], and may be bound via O or S, (viii) amino group optionally mono- or di-substituted with a C₁₋₃ alkyl group (e.g. methyl, ethyl, propyl, etc.), (ix) cyclic amino group optionally substituted with 1 to 3 of C₁₋₃ alkyl group (e.g., methyl, ethyl, etc.), benzyl, phenyl and the like (e.g., a 5- to 6-membered cyclic amino group which may contain an oxygen atom or a sulfur atom as ring constituent atoms in addition to a nitrogen atom of the cyclic amino group derived (by removing one hydrogen atom) from a cyclic amine such as piperidine, pyrrolidine, morpholine, thiomorpholine, piperazine, 4-methylpiperazine, 4-benzylpiperazine, 4-phenylpiperazine, 1,2,3,4-tetrahydroisoquinoline, phthalimide, etc.), (x) a 5- to 6-membered aromatic heterocyclic group which may contain 1 to 4 hetero atoms selected from N, O and S, and may be bound via O or S (e.g., pyridyl, imidazolyl, indolyl, tetrazolyl, etc.), (xi) a halogen atom (e.g., chlorine, fluorine, bromine, iodine, etc.), (xii) an C₁₋₄ alkyl group (e.g., methyl, ethyl, propyl, isopropyl, butyl, tert-butyl, etc.), C₁₋₄ alkoxy group (e.g., methoxy, ethoxy, propoxy, isopropoxy, butoxy, t-butoxy, etc.) or C₁₋₄ alkylthio group (e.g., methylthio, ethylthio, propylthio, isopropylthio, butylthio, tert-butylthio, etc.), each of which may be substituted with a substituent selected from C₁₋₄ alkoxy group, C₁₋₄ alkylthio group, carboxyl group and phenyl group, (xiii) a C₅₋₇ cycloalkyl group (e.g., cyclopentyl, cyclohexyl, cycloheptyl, etc.), and (xiv) a C₁₋₇ alkanoyloxy (e.g., formyloxy, acetoxy, propionyloxy, butyryloxy, t-butoxycarbonyloxy, isobutyryloxy, valeryloxy, pivaloyloxy, etc.). 1 to 6, preferably 1 to 3 of these substituents may be present at a substitutable position. In addition, two substituents may be combined to form C₃₋₆ alkylene, C₃₋₆ alkyleneoxy, C₃₋₆ alkylenedioxy or the like, for example, when two adjacent substituents on a phenyl group are combined to form C₄ alkylene, tetrahydronaphthalene group is formed.

Specific examples of the group represented by formula —X^{1c}—X^{2c}—Ar—X^{3c}—X^{4c}—COOH in R^{1c} include an optionally substituted (carboxy-heteroaryl)-C₁₋₄ alkyl group [preferably, an optionally substituted (carboxy-furyl)-C₁₋₄ alkyl group], an optionally substituted (carboxy-C₆₋₁₀ aryl)-C₁₋₄ alkyl group, an optionally substituted carboxy-heteroaryl group, an optionally substituted carboxy-C₆₋₁₀ aryl group, an optionally substituted (carboxy-C₁₋₄ alkyl)-heteroaryl group, an optionally substituted (carboxy-C₁₋₄ alkyl)-C₆₋₁₀ aryl group [preferably, a (carboxy-C₂₋₃ alkyl)-C₆₋₁₀ aryl group], an optionally substituted (carboxy-C₁₋₄ alkyl)-heteroaryl-C₁₋₄ alkyl group, an optionally substituted (carboxy-C₁₋₄ alkyl)-C₇₋₁₄ aralkyl group [preferably, an optionally substituted (carboxy-C₁₋₃ alkyl)-C₇₋₁₄ aralkyl group], an optionally substituted (carboxy-C₁₋₄ alkoxy)-C₆₋₁₀ aryl group, an optionally substituted (carboxy-C₁₋₄ alkoxy)-C₆₋₁₀ aryl-C₁₋₄ alkyl group, an optionally substituted (carboxy-C₁₋₄ alkyl)-C₆₋₁₀ aryloxy-C₁₋₄ alkyl group, an optionally substituted (carboxy-C₆₋₁₀ aryloxy)-C₁₋₄ alkyl group and an optionally substituted (carboxy-C₁₋₄ alkylthio)-heteroaryl group.

Herein, the same group as the aforementioned “aromatic heterocyclic group” may be exemplified for heteroaryl, and the heteroaryl may have the same substituent as the substituent which the aforementioned “aromatic heterocyclic group” may have. In addition, examples of C₆₋₁₀ aryl include phenyl, naphthyl, azulenyl, and phenyl is preferably used. The C₆₋₁₀ aryl may have the same substituent as the substituent which the aforementioned “aromatic heterocyclic group” may have.

Examples of the alkyl group in the optionally substituted (carboxyfuryl)-C₁₋₄ alkyl group represented by R¹ include C₁₋₄ linear or branched alkyl group such as methyl, ethyl, n-propyl, isopropyl, n-butyl, isobutyl, 1,1-dimethylethyl, and the like. Among these, a C₁₋₄ alkyl group such as methyl, ethyl, n-propyl, isopropyl and n-butyl are preferred, and methyl, ethyl and n-propyl are more preferred. Examples of the carboxyfuryl group include 3-carboxy-2-furyl, 4-carboxy-2-furyl, 2-carboxy-3-furyl, 2-carboxy-5-furyl and the like. Among these, 3-carboxy-2-furyl and 4-carboxy-2-furyl are preferred, and 3-carboxy-2-furyl is more preferred.

Examples of the C₂₋₃ alkyl group in the optionally substituted (carboxy-C₂₋₃ alkyl)-C₆₋₁₀ aryl group represented by R^{1c} include ethyl, n-propyl and isopropyl, and ethyl and n-propyl are preferred. Examples of the C₆₋₁₀ aryl group include phenyl, naphthyl and azulenyl, and phenyl is preferred.

Examples of the C₁₋₃ alkyl group in the optionally substituted (carboxy-C₁₋₃ alkyl)-C₇₋₁₄ aralkyl group represented by R^{1c} include methyl, ethyl, n-propyl and isopropyl, and methyl and ethyl are preferred, and ethyl is particularly preferred. Examples of a C₇₋₁₄ aralkyl group (a C₆₋₁₀ aryl-C₁₋₄ alkyl group) include phenylmethyl, 1-phenylethyl, 2-phenylethyl, 3-phenylpropyl, 2-phenylpropyl, 4-phenylbutyl, (1-naphthyl)methyl, (2-naphthyl)methyl, 1-(1-naphthyl)ethyl, 1-(2-naphthyl)ethyl, 3-(1-naphthyl)propyl, 3-(1-naphthyl)propyl, 4-(1-naphthyl)butyl and 4-(2-naphthyl)butyl, and phenylmethyl, 1-phenylethyl, 3-phenylpropyl, (1-naphthyl)methyl, (2-naphthyl)methyl, (1-naphthyl)ethyl and (2-naphthyl)ethyl are preferred, and phenylmethyl and 2-phenylethyl are particularly preferred.

As for the substituent in case that each group represented by R^{1c} has a substituent, the same as in the substituent which the “divalent aromatic ring group” in the “optionally substituted divalent aromatic ring group” represented by Ar may have may be exemplified, and 1 to 6, preferably 1 to 3 of these substituents can be present at substitutable positions. In addition, in each group represented by R^{1c}, it is preferable that the carboxylic portion is unsubstituted, and an arbitrary portion other than the carboxylic portion may have a substitutable substituent at a substitutable position.

As for R^{1c}, 3-carboxypropyl group, 1-carboxyethyl group, or a C₃₋₆ linear alkyl-sulfonyl group, a (carboxy-C₅₋₇ cycloalkyl)-C₁₋₃ alkyl group, a (carboxyfuryl)-alkyl group, a carboxy-C₆₋₁₀ aryl group, a (carboxy-C₁₋₄ alkyl)-C₆₋₁₀ aryl group [preferably, a (carboxy-C₂₋₃ alkyl)-C₆₋₁₀ aryl group], and a (carboxy-C₁₋₃ alkyl)-C₇₋₁₄ aralkyl group, each of which may have a substituent, and the like are preferred, an optionally substituted (carboxy-C₁₋₄ alkyl)-C₆₋₁₀ aryl group is preferred, and an optionally substituted (carboxy-C₂₋₃ alkyl)-C₆₋₁₀ aryl group is more preferred. In particular, an optionally substituted (carboxy-C₂₋₃ alkyl)-phenyl group is preferred.

Examples of the C₃₋₆ alkyl group in the C₃₋₆ alkyl group optionally substituted with an alkanoyloxy group or a hydroxy group represented by R^{2c} include n-propyl, isopropyl, 1,1-dimethylethyl, n-butyl, isobutyl, n-pentyl, 2,2-dimethylpropyl, isopentyl, n-hexyl, isohexyl and the like. Among these, isopropyl, 1,1-dimethylethyl, n-butyl, isobutyl, 2,2-dimethylpropyl and isohexyl are preferred, and 2,2-dimethylpropyl is particularly preferred.

Examples of the alkanoyloxy group in the C₃₋₆ alkyl group optionally substituted with an alkanoyloxy group or a hydroxy group represented by R^{2c} include a C₁₋₂₀ alkanoyloxy group such as formyloxy, acetoxy, propionyloxy, butyryloxy, tert-butoxycarbonyloxy, isobutyryloxy, valeryloxy, pivaloyloxy, lauryloxy, palmitoyloxy, stearoyloxy (preferably a C₁₋₇ alkanoyloxy group, etc.). Among these, acetoxy, propionyloxy, tert-butoxycarbonyloxy and palmitoyloxy are preferred, and acetoxy is particularly preferred. 1 to 3 of the alkanoyloxy groups or the hydroxy groups may substitute at a substitutable position.

Preferable examples of C₃₋₆ alkyl group optionally substituted with an alkanoyloxy group or a hydroxy group represented by R^{2c} include 2,2-dimethylpropyl, 3-hydroxy-2,2-dimethylpropyl, 3-hydroxy-2-hydroxymethyl-2-methylpropyl, 3-acetoxy-2,2-dimethylpropyl, 3-acetoxy-2-hydroxymethyl-2-methylpropyl and 3-acetoxy-2-acetoxymethyl-2-methylpropyl. Among these, 2,2-dimethylpropyl, 3-hydroxy-2,2-dimethylpropyl and 3-acetoxy-2,2-dimethylpropyl are particularly preferred.

In addition, as R^{2c}, a C₃₋₆ alkyl group having an alkanoyloxy group and/or hydroxy group is preferred.

Examples of the lower alkyl group represented by R^{3c} include a C₁₋₆ alkyl group such as methyl, ethyl, n-propyl, isopropyl, n-butyl, tert-butyl, pentyl, hexyl. Among these, a C₁₋₃ alkyl group is preferred. In view of the pharmacological activity, methyl group is particularly preferred as R^{3c}.

Examples of the halogen atom represented by W include chlorine, fluorine, bromine and iodine atom. Among these, chlorine atom is preferred.

The present invention includes the compound represented by the formula (Ic) in the form of either free or a pharmacologically acceptable salt thereof. As such salt, when the compound represented by the formula (Ic) has an acidic group such as carboxyl group, it may form a salt with an inorganic base (e.g., alkali metals such as sodium and potassium, alkaline earth metals such as calcium and magnesium, transition metals such as zinc, iron and copper, etc.) or an organic base (e.g., organic amines such as trimethylamine, triethylamine, pyridine, picoline, ethanolamine, diethanolamine, triethanolamine, dicyclohexylamine and N,N′-dibenzylethylenediamine, and basic amino acids such as arginine, lysine and ornithine, etc.)

In case where the compound represented by the formula (Ic) of the present invention has a basic group such as amino group, it may form a salt with inorganic acids or organic acids (e.g., hydrochloric acid, nitric acid, sulfuric acid, phosphoric acid, carbonic acid, bicarbonic acid, formic acid, acetic acid, propionic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid, etc.), and acidic amino acid such as aspartic acid, glutamic acid, and the like.

The compound represented by the formula (Ic) or a salt thereof has asymmetric carbon atoms at 3- and 5-position, but it may be in a mixture of the stereoisomers, and the isomers may also be separated by conventional means. The trans isomer wherein the substituents on 3- and 5-positions are directed in the opposite direction relative to the plane of the 7-membered ring is preferred, and in particular, the isomer wherein the absolute configuration at 3-position is R-configuration and the absolute configuration at 5-position is S-configuration is preferred. In addition, it may be a racemic compound or an optically active isomer. The optically active isomer can be separated from the racemic compound by a known optical resolution means.

As the compound represented by the formula (Ic) of the present invention or a salt thereof, the following compounds are preferred specifically.
N-propanesulfonyl-[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetamide or a salt thereof;
(2R)-2-[[(3R,5S)-7-chloro-5-(2,3-dimethoxyphenyl)-1-(2,2-dimethylpropyl)-2-oxo-1,2,3,5-tetrahydro-4,1-benzoxazepin-3-yl]acetyl]aminopropionic acid or a salt thereof;
3-[3-[[(3R,5S)-7-chloro-5-(2,3-dimethoxyphenyl)-1-(2,2-dimethylpropyl)-2-oxo-1,2,3,5-tetrahydro-4,1-benzoxazepin-3-yl]acetyl]aminophenyl]propionic acid or a salt thereof;
4-[[(3R,5S)-7-chloro-5-(2,3-dimethoxyphenyl)-1-(2,2-dimethylpropyl)-2-oxo-1,2,3,5-tetrahydro-4,1-benzoxazepin-3-yl]acetyl]aminobutanoic acid or a salt thereof;
trans-4-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]-aminomethyl-1-cyclohexane carboxylic acid or a salt thereof;
trans-4-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]-aminomethyl-1-cyclohexanecarboxylic acid or a salt thereof;
3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-fluorophenyl]propionic acid or a salt thereof;
3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methylphenyl]propionic acid or a salt thereof;
3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]amino]-4-methylphenyl]propionic acid or a salt thereof;
3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminomethyl]phenyl]propionic acid or a salt thereof;
3-[3-[[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]aminomethyl]phenyl]propionic acid or a salt thereof;
3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]propionic acid or a salt thereof;
2-[2-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]ethyl]furan-3-carboxylic acid or a salt thereof;
3-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-fluorophenyl]propionic acid or a salt thereof;
3-[3-[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]aminophenyl]propionic acid or a salt thereof;
4-[3-[[[(3R,5S)-7-chloro-5-(2,3-dimethoxyphenyl-1-(3-hydroxy-2,2-dimethylpropyl)-2-oxo-1,2,3,5-tetrahydro-4,1-benzoxazepin-3-yl]acetyl]amino]-4-methoxyphenyl]butanoic acid or a salt thereof;
5-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-methoxyphenyl]pentanoic acid or a salt thereof;
5-[3-[[[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl]amino]-4-fluorophenyl]pentanoic acid or a salt thereof.

The compound represented by the above-mentioned formula (Ic) or a salt thereof can be produced, for example, according to a method disclosed in EP A 567,026, WO95/21834 (international application based on Japanese Patent Application No. 6-15531), EP A 645,377 (application based on Japanese Patent Application No. 6-229159), EP A 645,378 (application based on Japanese Patent Application No. 6-229160), WO01/98282 (international application based on Japanese Patent Application No. 2000-190253) and the like, or analogous methods thereto.

As raw materials of the compound represented by the formula (I) of the present invention, the same salts as those mentioned above can be used, but they are not particularly limited as long as they do not interfere with the reaction.

Preferable example of each of the definition in the compound represented by formula (II) is as follows.

The substituent of the “optionally substituted benzene ring” represented by ring A includes halogen (e.g. fluorine, chlorine, bromine, iodine), an optionally substituted lower alkyl group having 1 to 4 carbon atoms (e.g. methyl, ethyl, propyl, butyl, tert-butyl etc.), an optionally substituted lower alkoxy group having 1 to 4 carbon atoms (e.g. methoxy, ethoxy, propoxy, isopropoxy, butoxy, tert-butoxy etc.), a hydroxyl group, a nitro group and cyano. The ring A may have 1 to 3, preferably 1 to 2 of these substituents. The adjacent substituents of these substituents may be taken together to form a ring. The substituent of the optionally substituted lower alkyl group having 1 to 4 carbon atoms or the optionally substituted lower alkoxy group having 1 to 4 carbon atoms includes halogen (e.g. fluorine, chlorine, bromine, iodine), and 1 to 3 substituents may be at optional substitutable positions. The ring A is preferably a benzene ring substituted with halogen atoms, etc., more preferably a benzene ring substituted with a chlorine atom. The ring A is preferably a benzene ring represented by the formula:

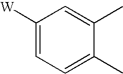

wherein W represents a halogen atom (e.g. fluorine, chlorine, bromine, iodine) and inter alia, W is preferably a chlorine atom.

The substituent of the “optionally substituted benzene ring” represented by ring B includes the same number of the same groups as those exemplified above as the substituent of the “optionally substituted benzene ring” represented by ring A. The ring B is preferably a benzene ring substituted with a lower alkoxy group having 1 to 4 carbon atoms, and inter alia, preferably a benzene ring represented by the formula:

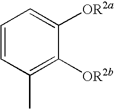

wherein R^{2a} and R^{2b} represent independently a hydrogen atom or a lower alkyl group having 1 to 4 carbon atoms (e.g. methyl, ethyl, propyl, butyl etc.) and particularly preferably R^{2a} and R^{2b} are both methyl groups.

The aromatic ring of the “optionally further substituted aromatic ring” represented by ring C includes an aromatic hydrocarbon ring and an aromatic heterocyclic ring. The aromatic hydrocarbon ring includes, for example, a benzene ring, a naphthalene ring and the like, and preferred is a benzene ring. The aromatic heterocyclic ring (the aromatic heterocyclic ring of the “optionally further substituted aromatic heterocyclic ring” represented by ring C′) includes, for example, an aromatic heterocyclic ring containing at least one (preferably 1 to 4, more preferably 1 to 2) of 1 to 3 kinds (preferably 1 or 2 kinds) of heteroatoms selected from an oxygen atom, a sulfur atom, a nitrogen atom and the like, as atoms constituting the ring system (ring atoms).

The aromatic heterocyclic ring includes 5- to 6-membered monocyclic aromatic heterocyclic rings such as furan, thiophene, pyrrole, oxazole, isoxazole, thiazole, isothiazole, imidazole, pyrazole, 1,2,3-oxadiazole, 1,2,4-oxadiazole, 1,3,4-oxadiazole, furazan, 1,2,3-thiadiazole, 1,2,4-thiadiazole, 1,3,4-thiadiazole, 1,2,3-triazole, 1,2,4-triazole, tetrazole, pyridine, pyridazine, pyrimidine, pyrazine, triazine and the like; 8- to 12-membered fused aromatic heterocyclic rings such as benzofuran, isobenzofuran, benzo[b]thiophene, indole, isoindole, 1H-indazole, benzimidazole, benzoxazole, 1,2-benzisoxazole, benzothiazole, benzopyrane, 1,2-benzisothiazole, 1H-benzotriazole, quinoline, isoquinoline, cinnoline, quinazoline, quinoxaline, phthalazine, naphthyridine, purine, pteridine, carbazole, α-carboline, β-carboline, γ-carboline, acridine, phenoxazine, phenothiazine, phenazine, phenoxathiine, thianthrene, phenanthridine, phenanthroline, indolizine, pyrrolo[1,2-b]pyridazine, pyrazolo[1,5-a]pyridine, imidazo[1,2-a]pyridine, imidazo[1,5-a]pyridine, imidazo[1,2-b]pyridazine, imidazo[1,2-a]pyrimidine, 1,2,4-triazolo[4,3-a]pyridine, 1,2,4-triazolo[4,3-b]pyridazine and the like (preferably a heterocyclic ring in which the aforementioned 5- to 6-membered monocyclic aromatic heterocyclic ring is fused with a benzene ring, or a heterocyclic ring in which the same or different two of the aforementioned 5- to 6-membered monocyclic aromatic heterocyclic rings are fused, more preferably a heterocyclic ring in which the aforementioned 5- to 6-membered monocyclic aromatic heterocyclic ring is fused with a benzene ring) and the like.

The ring C is preferably a monocyclic aromatic heterocyclic ring, a benzene ring or the like, and inter alia, preferred is a 5-membered monocyclic aromatic heterocyclic ring such as pyrazole, imidazole, thiazole, oxazole, isoxazole, 1,2,4-oxadiazole, 1,3,4-oxadiazole or the like.

Although the ring C may be an aromatic ring having a hydrogen atom that may be deprotonated or an aromatic ring having no hydrogen atom that may be deprotonated, an aromatic ring having no hydrogen atom that may be deprotonated is preferred. The aromatic ring having no hydrogen atom that may be deprotonated includes, in addition to an aromatic ring originally having no hydrogen atom that may be deprotonated (e.g. benzene ring, thiazole, oxazole, isoxazole, 1,2,4-oxadiazole, 1,3,4-oxadiazole etc.), an aromatic ring in which a hydrogen atom that may be deprotonated is substituted (e.g. pyrrole, pyrazole, imidazole etc. whose hydrogen atom on the ring-constituting nitrogen atom is substituted or which is bound to X^{1a} or/and X^{1b} via the ring-constituting nitrogen atom).

The substituent, which the aromatic ring of the “optionally further substituted aromatic ring” represented by ring C may have, includes (i) a carboxyl group optionally esterified with an optionally halogenated C₁₋₆ alkyl group or an optionally halogenated C₆₋₁₀ aryl-C₁₋₄ alkyl group (e.g. methyl, ethyl, propyl, isopropyl, butyl, tert-butyl, phenyl, benzyl etc.), (ii) a phosphoric acid group optionally mono- or di-substituted with optionally halogenated C₁₋₆ alkyl (e.g. methyl, ethyl, n-propyl, isopropyl, n-butyl, isobutyl, n-pentyl, isopentyl, neopentyl, hexyl etc.) or C₂₋₇ alkanoyloxy-C₁₋₆ alkyl such as acetoxymethyl or pivaloyloxymethyl, (iii) a sulfonic acid group, (iv) a sulfonamide group optionally substituted with an optionally halogenated C₁₋₆ alkyl group or an optionally halogenated C₆₋₁₀ aryl-C₁₋₄ alkyl group (e.g. methyl, ethyl, propyl, isopropyl, butyl, tert-butyl, benzyl etc.), (v) a hydroxyl group and a sulfhydryl group, which may be optionally substituted with an optionally halogenated C₁₋₃ alkyl group (e.g. methyl, ethyl, propyl etc.), (vi) a carbamoyl group, (vii) a phenyl group optionally substituted with 1 to 5 substituents [e.g. hydroxyl group, chlorine, fluorine, aminosulfonyl group, amino group optionally substituted with C₁₋₃ alkyl group (e.g. methyl, ethyl, propyl etc.)] and optionally bound to the aromatic ring via O or S, (viii) an amino group optionally mono- or di-substituted with an optionally halogenated C₁₋₃ alkyl group (e.g. methyl, ethyl, propyl etc.), (ix) a cyclic amino group optionally substituted with 1 to 3 C₁₋₃ alkyl (e.g. methyl, ethyl etc.), benzyl, phenyl and the like (e.g. a 5- to 6-membered cyclic amino group optionally containing an oxygen atom or a sulfur atom in addition to nitrogen atoms as ring-constituting atoms, such as a cyclic amino group derived (by removing one hydrogen atom) from cyclic amine such as piperidine, pyrrolidine, morpholine, thiomorpholine, piperazine, 4-methylpiperazine, 4-benzylpiperazine, 4-phenylpiperazine, 1,2,3,4-tetrahydroisoquinoline or phthalimide), (x) a 5- to 6-membered aromatic heterocyclic group containing 1 to 4 heteroatoms selected from N, C and S and optionally bound to the aromatic ring via O or S (e.g. pyridyl, imidazolyl, indolyl, tetrazolyl etc.), (xi) a halogen atom (e.g. chlorine, fluorine, bromine, iodine etc.), (xii) a C₁₋₄ alkyl group (e.g. methyl, ethyl, propyl, isopropyl, butyl, tert-butyl etc.), a C₁₋₄ alkoxy group (e.g. methoxy, ethoxy, propoxy, isopropoxy, butoxy, tert-butoxy etc.) or a C₁₋₄ alkylthio group (e.g. methylthio, ethylthio, propylthio, isopropylthio, butylthio, tert-butylthio etc.), each of which may be optionally substituted with a substituent selected from a halogen atom, a C₁₋₄ alkoxy group, a C₁₋₄ alkylthio group, carboxyl and phenyl, (xiii) a C₅₋₇ cycloalkyl group (e.g. cyclopentyl, cyclohexyl, cycloheptyl etc.), and (xiv) optionally halogenated C₁₋₇ alkanoyloxy (e.g. formyloxy, acetoxy, propionyloxy, butyryloxy, tert-butoxycarbonyloxy, isobutyryloxy, valeryloxy, pivaloyloxy etc.). The “optionally further substituted aromatic ring” may be substituted with 1 to 6, preferably 1 to 3 such substituents at substitutable positions. Two of such substituents may be taken together to form C₃₋₆ alkylene, C₃₋₆ alkyleneoxy, C₃₋₆ alkylenedioxy or the like. For example, when two adjacent substituents on a phenyl group are linked each other to form C₄ alkylene, a tetrahydronaphthyl group is formed.

The lower alkyl group of the “lower alkyl group optionally substituted with an optionally substituted hydroxyl group” represented by R¹ includes, for example, C₁₋₆ alkyl such as methyl, ethyl, n-propyl, isopropyl, n-butyl, isobutyl, n-pentyl, isopentyl, neopentyl, hexyl and the like. Among them, a C₃₋₆ alkyl group is preferred and a C₄₋₅ alkyl group is more preferred. Inter alia, a branched C₄₋₅ alkyl group such as isobutyl, neopentyl or the like is preferred.

The substituent, which the lower alkyl group of the “lower alkyl group optionally substituted with an optionally substituted hydroxyl group” represented by R¹ may have, includes a hydroxyl group optionally substituted with C₂₋₂₀ alkanoyl or C₁₋₇ alkyl. Such substituent includes, for example, a hydroxyl group, acetyloxy (acetoxy), propionyloxy, tert-butoxycarbonyloxy, palmitoyloxy, dimethylaminoacetyloxy, 2-aminopropionyloxy and the like. The lower alkyl group may be substituted with 1-3 such substituents at substitutable positions.

Examples of R¹ include 1-propyl, 1-isopropyl, 1-isobutyl, 1-neopentyl, 2,2-dimethyl-3-hydroxypropyl, 3-hydroxy-2-hydroxymethyl-2-methylpropyl, 3-acetoxy-2,2-dimethylpropyl, 3-acetoxy-2-hydroxymethyl-2-methyl-propyl, 3-acetoxy-2-acetoxymethyl-2-methylpropyl, [1-(hydroxymethyl)cyclobutyl]methyl and the like. Among them, preferred are 2,2-dimethyl-3-hydroxypropyl, 3-hydroxy-2-hydroxymethyl-2-methylpropyl, 3-acetoxy-2,2-dimethylpropyl, 3-acetoxy-2-hydroxymethyl-2-methylpropyl, 3-acetoxy-2-acetoxymethyl-2-methylpropyl and the like.

The lower alkylene of the “optionally substituted lower alkylene” represented by X^{1a} includes, for example, C₁₋₆ alkylene such as methylene, dimethylene, trimethylene, tetramethylene, pentamethylene, hexamethylene and the like. Among them, preferred is straight chain C₁₋₄ alkylene such as methylene, dimethylene, trimethylene, tetramethylene or the like, and more preferred is straight chain C₁₋₃ alkylene.

The substituent, which the lower alkylene of the “optionally substituted lower alkylene” represented by X^{1a} may have, includes the same groups as those exemplified above as the substituent, which the aromatic ring of the “optionally further substituted aromatic ring” represented by ring C may have, an oxo group and the like. The “lower alkylene” may be substituted with 1 to 6, preferably 1 to 3 such substituents at substitutable positions.

X^{1a} is preferably a bond or straight chain C₁₋₃ alkylene, and particularly preferably methylene.

The lower alkylene of the “optionally substituted lower alkylene” represented by X^{1b} includes the same groups as those exemplified as the lower alkylene of the “optionally substituted lower alkylene” represented by X^{1a}. The substituent, which the lower alkylene of the “optionally substituted lower alkylene” represented by X^{1b} may have, includes the same number of the same groups as those exemplified as the substituent which the lower alkylene of the “optionally substituted lower alkylene” represented by X^{1a} may have.

X^{1b} is preferably a bond or straight chain C₁₋₃ alkylene, and particularly preferably a bond.

X² is preferably a bond.

The “divalent hydrocarbon group” of the “optionally substituted divalent hydrocarbon group” represented by X³ includes a group formed by removing one hydrogen atom from a hydrocarbon group. The hydrocarbon group includes a C₁₋₇ straight or branched chain alkyl group (e.g. methyl, ethyl, n-propyl, isopropyl, n-butyl, isobutyl, 1,1-dimethylethyl, n-pentyl, 3-methylbutyl, 2-methylbutyl, 1-methylbutyl, 1-ethylpropyl, n-hexyl, 4-methylpentyl, 3-methylpentyl, 2-methylpentyl, 2-ethylbutyl, 1-ethylbutyl, neopentyl, hexyl, heptyl), a C₃₋₇ cycloalkyl group (cyclopropyl, cyclobutyl, cyclopentyl, cyclohexyl, cyclohexylmethyl etc.), a straight or branched chain C₂₋₆ alkenyl group (e.g. vinyl, allyl, isopropenyl, 2-methylallyl, 1-propenyl, 2-methyl-1-propenyl, 2-methyl-2-propenyl, 1-butenyl, 2-butenyl, 3-butenyl, 2-ethyl-1-butenyl, 2-methyl-2-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl, 5-hexenyl etc.), a C₆₋₁₀ aryl group (e.g. phenyl, naphthyl), a C₇₋₁₄ arylalkyl group (e.g. benzyl, phenethyl, naphthylmethyl) and the like.

The substituent, which the “divalent hydrocarbon group” of the “optionally substituted divalent hydrocarbon group” represented by X³ may have, includes the same group as those exemplified above as the substituent which the lower alkylene of the “optionally substituted lower alkylene” represented by X^{1a} may have, optionally halogenated C₁₋₆ alkylidene (e.g. methylidene, ethylidene, propylidene, isopropylidene, butenylidene etc.), vinylidene, cyclohexylidene, benzylidene and the like. The “divalent hydrocarbon group” may be substituted with 1 to 6, preferably 1 to 3 such substituents at substitutable positions.

The “divalent hydrocarbon group” of the “optionally substituted divalent hydrocarbon group” represented by X³ preferably includes (1) straight or branched chain alkylene in which the number of carbon atoms constituting the straight chain part is 1 to 7 (preferably 1 to 4) (e.g. methylene, ethylene, trimethylene, tetramethylene, pentamethylene, hexamethylene, heptamethylene, propylene, ethylmethylene, ethylethylene, propylethylene, butylethylene, methyltetramethylene, methyltrimethylene etc.), (2) a double bond-containing carbon chain in which the number of carbon atoms constituting the straight chain part is 2 to 7 (preferably 2 to 4) (e.g. vinylene, propenylene, butenylene, butadienylene, methylpropenylene, ethylpropenylene, propylpropenylene, methylbutenylene, ethylbutenylene, propylbutenylene, methylbutadienylene, ethylbutadienylene, propylbutadienylene, pentenylene, hexenylene, heptenylene, pentadienylene, hexadienylene, heptadienylene etc.), (3) phenylene (e.g. 1,2-phenylene, 1,3-phenylene, 1,4-phenylene etc.) and (4) a divalent group in which phenylene and alkylene and/or alkenylene are combined (e.g. —CH₂—C₆H₄—, —CH₂CH₂—C₆H₄—, —CH₂—C₆H₄—CH₂— etc.).

X³ is preferably C₁₋₄ alkylene such as methylene, ethylene, trimethylene, tetramethylene or the like, vinylene, propenylene, phenylene or the like.

The “optionally esterified or amidated carboxyl group” represented by Y includes carboxyl, lower alkoxycarbonyl having 2 to 7 carbon atoms (e.g. methoxycarbonyl, ethoxycarbonyl, propoxycarbonyl, isopropoxycarbonyl, butoxycarbonyl, tert-butoxycarbonyl, sec-butoxycarbonyl, pentyloxycarbonyl, isopentyloxycarbonyl, neopentyloxycarbonyl etc.), C₇₋₁₄ aryloxycarbonyl (e.g. phenoxycarbonyl, 1-naphthoxycarbonyl), C₈₋₁₂ aralkyloxycarbonyl (e.g. benzyloxycarbonyl etc.), carbamoyl, N—C₁₋₆ alkylcarbamoyl, N,N-diC₁₋₆ alkylcarbamoyl, N—C₈₋₁₂ aralkylcarbamoyl, N,N-diC₈₋₁₂ aralkylcarbamoyl, 1-pyrrolidinylcarbonyl, piperidinocarbonyl, morpholinocarbonyl and the like. Among them, Y is preferably carboxyl, methoxycarbonyl, ethoxycarbonyl or the like, and particularly preferably carboxyl.

The compound represented by the formula (II) may be in a free form or a pharmacologically acceptable salt form, and both forms are included in the scope of the present invention. When the compound represented by the formula (II) has an acidic group such as a carboxyl group, etc., the compound may form a salt with inorganic bases (e.g. alkali metal such as sodium, potassium etc., alkaline earth metal such as calcium, magnesium etc., transition metal such as zinc, iron, copper etc.) or organic bases (e.g. organic amines such as trimethylamine, triethylamine, pyridine, picoline, ethanolamine, diethanolamine, triethanolamine, tris(hydroxymethyl)methylamine, dicyclohexylamine, N,N′-dibenzylethylenediamine and t-butylamine, basic amino acids such as arginine, lysine ornithine, and the like).

When the compound represented by the formula (II) of the present invention has a basic group such as an amino group or the like, the compound may form a salt with inorganic acids or organic acids (e.g. hydrochloric acid, nitric acid, sulfuric acid, phosphoric acid, carbonic acid, bicarbonic acid, formic acid, acetic acid, propionic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid, etc.) or acidic amino acids such as aspartic acid or glutamic acid.

The compound represented by the formula (II) or a salt thereof has asymmetric carbon atoms at the 3-position and the 5-position, and may be a mixture of stereoisomers. The isomers may be separated by a known means. Preferred is the trans form in which the substituents at the 3-position and 5-position are directed to the opposite direction each other relative to the plane of the 7-membered ring, and specifically preferred is a compound having the absolute configuration represented by the formula (IIa). In addition, the compound represented by the formula (II) or a salt thereof may be a racemic form or an optically active form, and the optically active form can be separated from the racemic form by a known optical resolution means.

As the compounds represented by formula (II) of the present invention or a salt thereof, specifically the followings are preferred.

(1) the compound wherein X^{1b} is a bond and Y is an optionally esterified carboxyl group;
(2) the compound wherein ring A is a benzene ring substituted with halogen atom(s);
(3) the compound wherein ring B is a benzene ring substituted with lower alkoxy group(s);
(4) the compound wherein ring C is an optionally further substituted monocyclic aromatic heterocyclic ring;
(5) the compound wherein ring C is an optionally further substituted benzene ring;
(6) the compound wherein ring C is an optionally further substituted aromatic ring having no hydrogen atom that may be deprotonated;
(7) the compound wherein X^{1a} is C₁₋₃ alkylene;
(8) the compound wherein X² is a bond;
(9) the compound wherein X³ is C₁₋₄ alkylene;
(10) the compound wherein the formula (II) is the formula (IIa):

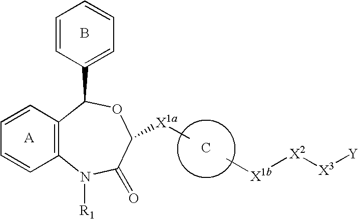

wherein respective symbols are as defined in the formula (II);
(11) 3-(2-{3-[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]propyl}-1,3-thiazol-5-yl)propionic acid, 3-(2-{2-[(3R,5S)-7-chloro-5-(2,3-dimethoxyphenyl)-1-(2,2-dimethylpropyl)-2-oxo-1,2,3,5-tetrahydro-4,1-benzoxazepin-3-yl]ethyl}-1,3-thiazol-4-yl)propionic acid, or a salt thereof;
(12) (2-{[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]methyl}-1,3-oxazol-5-yl)propionic acid, (2-{[(3R,5S)-7-chloro-5-(2,3-dimethoxyphenyl)-1-isobutyl-2-oxo-1,2,3,5-tetrahydro-4,1-benzoxazepin-3-yl]methyl}-1,3-oxazol-5-yl)acetic acid, or a salt thereof;
(13) 5-(3-{[(3R,5S)-7-chloro-5-(2,3-dimethoxyphenyl)-1-(2,2-dimethylpropyl)-2-oxo-1,2,3,5-tetrahydro-4,1-benzoxazepin-3-yl]methyl}-1,2,4-oxadiazol-5-yl)pentanoic acid, 5-(3-{[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]methyl}-1,2,4-oxadiazol-5-yl)pentanoic acid, 5-(3-{[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]methyl}-1,2,4-oxadiazol-5-yl)pentanoic acid, 4-{[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl}phenyl)acetic acid, or a salt thereof;

The compounds represented by formula (II) can be produced by a method disclosed in, for example, WO 2005/012272.

Among the squalene synthase inhibitors mentioned above, the following is particularly preferred:
(1) N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid,
(2) 3-(2-{3-[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]propyl}-1,3-thiazol-5-yl)propionic acid,
(3) 3-(2-{2-[(3R,5S)-7-chloro-5-(2,3-dimethoxyphenyl)-1-(2,2-dimethylpropyl)-2-oxo-1,2,3,5-tetrahydro-4,1-benzoxazepin-3-yl]ethyl}-1,3-thiazol-4-yl)propionic acid,
(4) (2-{[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]methyl}-1,3-oxazol-5-yl)propionic acid,
(5) (2-{[(3R,5S)-7-chloro-5-(2,3-dimethoxyphenyl)-1-isobutyl-2-oxo-1,2,3,5-tetrahydro-4,1-benzoxazepin-3-yl]methyl}-1,3-oxazol-5-yl)acetic acid,
(6) 5-(3-{[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]methyl}-1,2,4-oxadiazol-5-yl)pentanoic acid,
(7) 5-(3-{[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]methyl}-1,2,4-oxadiazol-5-yl)pentanoic acid,
(8) 5-(3-{[(3R,5S)-7-chloro-5-(2,3-dimethoxyphenyl)-1-(2,2-dimethylpropyl)-2-oxo-1,2,3,5-tetrahydro-4,1-benzoxazepin-3-yl]methyl}-1,2,4-oxadiazol-5-yl)pentanoic acid, and
(9) (4-{[(3R,5S)-7-chloro-5-(2,3-dimethoxyphenyl)-1-(3-hydroxy-2,2-dimethylpropyl)-2-oxo-1,2,3,5-tetrahydro-4,1-benzoxazepin-3-yl]acetyl}phenyl)acetic acid.

The present invention can be carried out with following administration forms.

The administration mode of a combination of a squalene synthase inhibitor (SSI) and a HMG-CoA reductase inhibitor (administration in combination) is not particularly limited, as long as the SSI and the HMG-CoA reductase inhibitor are in combination at the time of administration. Examples of such administration mode include:

(1) administration of a single preparation obtained by formulating the SSI and the HMG-CoA reductase inhibitor simultaneously;
(2) simultaneous administration via the same administration route of two kinds of preparations obtained by formulating the SSI and the HMG-CoA reductase inhibitor respectively;
(3) separate administration at an interval via the same administration route of two kinds of preparations obtained by formulating the SSI and the HMG-CoA reductase inhibitor respectively;
(4) simultaneous administration via different route of two kinds of preparations obtained by formulating the SSI and the HMG-CoA reductase inhibitor respectively;
(5) separate administration at an interval via different administration route of two kinds of preparations obtained by formulating the SSI and the HMG-CoA reductase inhibitor respectively (for example, administration of the SSI and then the HMG-CoA reductase inhibitor in the subsequent order or in the reverse order).

Dosage of the HMG-CoA reductase inhibitor can be appropriately selected on the basis of the clinically used dosage. The combination ratio of the SSI and the HMG-CoA reductase inhibitor can be appropriately selected depending on a subject to be administered, an administration route, targeted diseases, symptoms, combinations thereof or the like. For example, if the subject to be administered is a human, although it depends on kinds of the HMG-CoA reductase inhibitor to be used, the SSI in the amount of 0.1 to 100 parts by weight (preferably, 0.5 to 100 parts by weight) (in the case of using atorvastatin as the HMG-CoA reductase inhibitor, the SSI in the amount of 0.1 to 10 parts by weight, more preferably 0.5 to 10 parts by weight) based on 1 part by weight of the HMG-CoA reductase inhibitor may be used.

When carrying out the above-mentioned invention, a pharmaceutical composition can be administered in a form of preparation which is prepared by a conventional method using conventional carriers for formulation in suitable amount, which carriers are suitably selected from, for example, an excipient (for example, calcium carbonate, kaolin, sodium hydrocarbonate, lactose, starches, crystalline cellulose, talc, granulated sugar, porous substances, etc.), a binder (for example, dextrin, gums, alcoholated starch, gelatin, hydroxypropylcellulose, hydroxypropylmethylcellulose, Pullulan, etc.), a disintegrating agent (for example, carboxymethylcellulose calcium, croscamellose sodium, crospovidone, low-substituted hydroxypropylcellulose, partially pregelatinated starch, etc.), a lubricant (for example, magnesium stearate, calcium stearate, talc, starch, sodium benzoate, etc.), a colorant (for example, tar dye, caramel, iron sesquioxide, titanium oxide, riboflavins, etc.), a taste-masking agent (for example, sweeters, flavors, etc.), a stabilizer (for example, sodium sulphite, etc.), a preservative (for example, parabens, sorbic acid, etc.) and the like. The pharmaceutical preparations of the present invention comprising the above-mentioned ones contain the SSI and/or the HMG-CoA reductase inhibitor in an effective amount for treatment and prevention of the disease. Further, the preparations used in the present invention may contain other drug ingredients as active ingredients than the SSI and/or the HMG-CoA reductase inhibitor. Such ingredient is not particularly limited as long as the object of the present invention is achieved, and can be used in a suitable mixing ratio. Specific examples of the preparation include tablets (including sugar-coated tablets, film-coated tablets, layered tablets), pills, capsules, granules, fine-granules, powders, syrup, emulsion, suspension, injection, suspended injection, inhaler, ointment, and the like. These preparations may be a release-controlled preparation (e.g., sustained-release microcapsules) such as quick-release preparations and sustained-release preparations. Among these preparations, oral preparations having advantages in convenience or compliance are preferred in some cases. When these preparations are a solid preparation, marks or characters for distinction may be printed on them or a cleavage line for division may be appended.

These preparations are prepared by a conventional method (for example, a method described in Japan Pharmacopoeia).

In the practice of the present invention, when a solid preparation (so-called fixed dose combination) containing both ingredients of SSI and HMG-CoA reductase inhibitor is used, it is produced, for example, according to the following production method.

1) After a SSI and a HMG-CoA reductase inhibitor are mixed together with additives such as excipients, the mixture is granulated while sprayed with a dispersion or solution of additives such as binders in a solvent (e.g. water). The resulting granules are mixed with additives such as disintegrants and lubricants and then, if necessary, compressed to produce a solid preparation.

2) After a SSI is mixed with additives such as excipients, the mixture is granulated while sprayed with a dispersion or solution of a HMG-CoA reductase inhibitor, and additives such as binders in a solvent (e.g. water). The resulting granules are mixed with additives such as disintegrants and lubricants and then, if necessary, compressed to produce a solid preparation.

3) After a SSI is mixed with additives such as excipients, the mixture is granulated while sprayed with a dispersion or solution of additives such as binders in a solvent (e.g. water).

On the other hand, after a HMG-CoA reductase inhibitor is mixed with additives such as excipients, the mixture is granulated while sprayed with a dispersion or solution of and additives such as binders in a solvent (e.g. water).

The granules containing SSI and the granules containing HMG-CoA reductase inhibitor thus obtained are mixed together with additives such as disintegrants and lubricants and then, if necessary, compressed to produce a solid preparation.

4) After a SSI is mixed with additives such as excipients, the mixture is granulated while sprayed with a dispersion or solution of additives such as binders in a solvent (e.g. water).

On the other hand, additives such as excipients are sprayed to granulate with a dispersion or solution of a HMG-CoA reductase inhibitor, and additives such as binders in a solvent (e.g. water).

The granules containing SSI and the granules containing HMG-CoA reductase inhibitor thus obtained are mixed together with additives such as disintegrants and lubricants and then, if necessary, compressed to produce a solid preparation.

5) After a SSI is mixed with additives such as excipients, the mixture is granulated while sprayed with a dispersion or solution of additives such as binders in a solvent (e.g. water). The resulting granules are mixed with additives such as disintegrants and lubricants to obtain mixed powder.

On the other hand, additives such as excipients are sprayed to granulate with a dispersion or solution of a HMG-CoA reductase inhibitor, and additives such as binders in a solvent (e.g. water). The resulting granules are mixed with additives such as disintegrants and lubricants to obtain mixed powder.

The mixed powder containing SSI and the mixed powder containing HMG-CoA reductase inhibitor thus obtained are layered and then compressed to produce a solid preparation (a two-layered tablet).

6) After a SSI is mixed with additives such as excipients, the mixture is granulated while sprayed with a dispersion or solution of additives such as binders in a solvent (e.g. water). The resulting granules are mixed with additives such as disintegrants and lubricants and then compressed into a core-tablet.

On the other hand, additives such as excipients are sprayed to granulate with a dispersion or solution of a HMG-CoA reductase inhibitor, and additives such as binders in a solvent (e.g. water). The resulting granules are mixed with additives such as disintegrants and lubricants to obtain mixed powder.

The thus obtained mixed powders are compressed as an outer layer on the above-described core-tablet to produce a solid preparation (a dry-coated tablet).

7) After a SSI is mixed with additives such as excipients, the mixture is granulated while sprayed with a dispersion or solution of additives such as binders in a solvent (e.g. water). The resulting granules are mixed with additives such as disintegrants and lubricants and then compressed into a tablet. This tablet is coated with a film solution of HMG-CoA reductase inhibitor, a coating base and additives such as light-blocking agents to produce a solid preparation (a film-coated tablet).

Dosage of the preparation of the present invention is varied depending on the administration route, symptoms and age or weight of patients, or the like. In the case of oral administration to an adult patient, it is preferable to administer 1 to 100 mg/day as the SSI or the HMG-CoA reductase inhibitor once or in two or more divided portions. The administration route may be via oral or non-oral.

### EXAMPLES

In the following, excellent effects of use in combination of Compound X as the representative compound of SSI and some HMG-CoA reductase inhibitors will be explained by describing specific pharmacological test results. However, these are examples of the combination effects of the SSI and HMG-CoA reductase inhibitor of the present invention, and so this combination effect is not limited to the following concrete pharmacological effects.

### Example 1

### Lowering Effect on Plasma Triglyceride by Use in Combination of Compound X and Atorvastatin

### Test Method:

To Wistar Fatty rats (19 week-old female, N=6), 10-mL/kg dose of the vehicle, Compound X (30 mg/kg) alone, atorvastatin (30 mg/kg) alone or combination of both drugs was administered orally for 8 days, once a day. On the morning of the following day after the 8^{th} administration, blood was collected under overnight fasting, and the concentration of plasma triglyceride was measured.

### Test Result:

| | Plasma triglyceride |
|---|---|
| Treatment | value (mg/dL) |
| Vehicle | 519.7 ± 43.5 |
| Compound X (30 mg/kg) | 376.5 ± 22.0 |
| atorvastatin (30 mg/kg) | 192.1 ± 15.2 |
| Combination of atorvastatin | 142.9 ± 15.3 |
| (30 mg/kg) + Compound X | |
| (30 mg/kg) | |

Average ± standard error (N = 6)

### Conclusion:

By use in combination of Compound X and atorvastatin, an additional action of lowering plasma triglyceride was observed (P<0.01, two-way ANOVA method).

### Example 2

### Lowering Effect on Plasma Cholesterol by Use in Combination of Compound X and Atorvastatin

### Test Method:

To Hartley guinea pigs (5 week-old male, N=12), RC-4 diet containing 0.05% cholesterol and 10% corn oil was loaded for 3 weeks, and was administered orally for 14 days, once a day, a 10-mL/kg dose of the vehicle, Compound X (30 mg/kg) alone, atorvastatin (3, 10, 30 mg/kg) alone or combination of atorvastatin (3, 10, 30 mg/kg) and Compound X (30 mg/kg). On the morning of the following day after the 14^{th} administration, blood was collected, and the total cholesterol in plasma was measured.

### Test Result:

| | Total cholesterol |
|---|---|
| Treatment | level (mg/dL) |
| Vehicle | 56.3 ± 4.1 |
| Compound X (30 mg/kg) | 44.4 ± 4.1 |
| Atorvastatin (3 mg/kg) | 46.1 ± 3.3 |
| Atorvastatin (10 mg/kg) | 37.4 ± 4.1 |
| Atorvastatin (30 mg/kg) | 33.5 ± 2.8 |
| Combination of atorvastatin | 39.2 ± 2.3 |
| (3 mg/kg) + Compound X | |
| (30 mg/kg) | |
| Combination of atorvastatin | 31.0 ± 3.7 |
| (10 mg/kg) + Compound X | |
| (30 mg/kg) | |
| Combination of atorvastatin | 27.9 ± 1.8 |
| (30 mg/kg) + Compound X | |
| (30 mg/kg) | |

Average ± standard error (N = 12)

### Conclusion:

By use in combination of Compound X and atorvastatin, an additional action of lowering the total cholesterol in plasma was observed (P<0.01, two-way ANOVA method).

### Example 3

### Lowering Effects on Plasma Cholesterol by Use in Combination of Compound X and Simvastatin

### Test Method:

To Hartley guinea pigs (5 week-old male, N=12), RC-4 diet containing 0.05% cholesterol and 10% corn oil was loaded for 3 weeks, and was administered orally for 14 days, once a day, a 10-mL/kg dose of the vehicle, Compound X (30 mg/kg) alone, simvastatin (10, 30, 100 mg/kg) alone or combination of simvastatin (10, 30, 100 mg/kg) and Compound X (30 mg/kg) On the morning of the following day after the 14^{th} administration, blood was collected, and the total cholesterol in plasma was measured.

### Test Result:

| | Total cholesterol |
|---|---|
| Treatment | level (mg/dL) |
| Vehicle | 56.9 ± 4.8 |
| Compound X (30 mg/kg) | 40.2 ± 4.7 |
| Simvastatin (10 mg/kg) | 41.0 ± 3.6 |
| Simvastatin (30 mg/kg) | 43.4 ± 2.2 |
| Simvastatin (100 mg/kg) | 31.7 ± 2.6 |
| Simvastatin (10 mg/kg) + | 40.0 ± 3.0 |
| Compound X (30 mg/kg) | |
| Simvastatin (30 mg/kg) + | 34.8 ± 2.3 |
| Compound X (30 mg/kg) | |
| Simvastatin (100 mg/kg) + | 24.2 ± 1.8 |
| Compound X (30 mg/kg) | |

Average ± standard error (N = 12)

### Conclusion:

By use in combination of Compound X and simvastatin, an additional action of lowering the total cholesterol in plasma was observed (P<0.01, two-way ANOVA method).

### Example 4

### Influence of Compound X and Atorvastatin on Liver Pathological Change

### Test Method:

To Hartley guinea pigs (5 week-old male, N=12) RC-4 diet containing 0.05% cholesterol and 10% corn oil was loaded for 3 weeks, and was administered orally for 14 days, once a day, a 10-mL/kg dose of the vehicle, Compound X (30 mg/kg) alone, atorvastatin (30 mg/kg) alone or combination of atorvastatin (30 mg/kg) and Compound X (30 mg/kg). On the morning of the following day after the 14^{th} administration, blood was collected, and the total cholesterol in plasma was measured. Furthermore, the liver was removed and pathological evaluation was conducted.

### Test Result:

| | Total | |
|---|---|---|
| | cholesterol | Centrilobular single |
| Treatment | (mg/dL) | liver cell necrosis |
| Vehicle | 53.6 ± 3.0 | 0 case in 12 cases |
| Compound X (30 mg/kg) | 40.1 ± 3.1 | 0 case in 12 cases |
| Atorvastatin(30 mg/kg) | 32.9 ± 1.5 | 3 cases in 12 cases |
| Atorvastatin(30 mg/kg) + | 28.3 ± 1.3 | 0 case in 12 cases |
| Compound X (30 mg/kg) | | |

Average ± standard error (N = 12)

### Conclusion:

Image of the liver cell necrosis was observed in the atorvastatin treatment group whereas it was not recognized in the group of Compound X alone or the group of Compound X and atorvastatin in combination.

### Example 5

### Influence of Compound X and Atorvastatin on Change of Liver-Deviated Enzymes

### Test Method:

To Hartley guinea pigs (5 week-old male, N=12), RC-4 diet containing 0.05% cholesterol and 10% corn oil was loaded for 2 weeks, and was administered orally for 7 days, once a day, a 5-mL/kg dose of the vehicle, atorvastatin (50 mg/kg) alone or combination of atorvastatin (50 mg/kg) and Compound X (30 mg/kg). On the morning of the following day after the 7^{th} administration, blood was collected, and the concentrations of the alanine aminotransferase and the aspartate aminotransferase, which are the markers of hepatic toxicity, in plasma were measured.

### Test Result:

| | Aspartate | Alanine |
|---|---|---|
| | aminotransferase | aminotransferase |
| Treatment | (I.U./L) | (I.U./L) |
| Vehicle | 43.0 ± 4.5 | 30.2 ± 1.2 |
| Atorvastatin (50 mg/kg) | 169.0 ± 39.1* | 48.9 ± 9.7 |
| Atorvastatin (50 mg/kg) + | 75.2 ± 8.1# | 30.7 ± 1.2 |
| Compound X (30 mg/kg) | | |

Average ± standard error (N = 12)
*P < 0.05 vs. Vehicle group (Student t-test).
#P < 0.05 vs. Atorvastatin Treatment group (Student t-test).

### Conclusion:

Significant increase in the aspartate aminotransferase in plasma was observed in the atorvastatin treatment group. However, the increase in the aminotransferase was not recognized in the group of Compound X and atorvastatin in combination.

### Example 6

### Influence of Compound X and Cerivastatin on Change of Muscle-Deviated Enzymes

### Test Method:

To Hartley guinea pigs (5 week-old male, N=16), a 5-mL/kg dose of the vehicle, cerivastatin (1 mg/kg) alone or combination of cerivastatin (1 mg/kg) and Compound X (30 mg/kg) was administered orally for 14 days, once a day. On the morning of the following day after the 14^{th} administration, blood was collected creatinine phosphokinase (CK) and myoglobin (Mb), which are the markers of muscle toxicity, in plasma was measured.

### Test Result:

| | TC | CK | Mb |
|---|---|---|---|
| Treatment | (mg/dL) | (mg/dL) | (ng/mL) |
| Vehicle | 24.2 ± 1.0 | 389 ± 99 | 0.3 ± 0.2 |
| Cerivastatin | 13.2 ± 0.8** | 4072 ± 800** | 25.7 ± 2.6** |
| (1 mg/kg) | | | |
| Cerivastatin | 12.0 ± 0.6 | 498 ± 62## | 1.1 ± 0.4## |
| (1 mg/kg) + | | | |
| Compound X | | | |
| (30 mg/kg) | | | |

Average ± standard error (N = 16).
**P < 0.01 vs. vehicle, and
##P < 0.01 vs. cerivastatin monotherapy by Student t-test.

### Conclusion:

Significant increases in the creatinine phosphokinase and myoglobin in plasma were observed in the cerivastatin treatment group. However, Compound X significantly ameliorated the increases in the creatinine phosphokinase and myoglobin induced by cerivastatin alone, with slightly increasing the cholesterol-lowering effect.

As is clear from the above, by a combined use of SSI and HMG-CoA reductase inhibitor, a superior prophylactic and/or therapeutic effect of hyperlipidemia can be obtained with covering the shortcomings of HMG-CoA reductase inhibitor, which cannot be obtained by a single administration.

The present inventors found out that by further using Ezetimibe as third ingredient together, more superior prophylactic and/or therapeutic effect of hyperlipidemia can be obtained.

Hereinafter, this three-drug combinational effect will be demonstrated by showing test results.

### Example 7

### Lowering Effect on Plasma Cholesterol by Use in Triple Combination of Compound X, Simvastatin and Ezetimibe

### Test Method:

To Hartley guinea pigs (5 week-old male, N=6), RC-4 diet containing 0.05% cholesterol and 10% corn oil was loaded for 3 weeks, and was administered orally for 14 days, once a day, a 6-mL/kg dose of the vehicle, Compound X (30 mg/kg) alone, combination of simvastatin (30 mg/kg) and ezetimibe (0.15 mg/kg) or triple combination of simvastatin (30 mg/kg), ezetimibe (0.15 mg/kg) and Compound X (30 mg/kg). On the morning of the following day after the 14^{th} administration, blood was collected, and the total cholesterol in plasma was measured.

### Test Result:

| | Total cholesterol |
|---|---|
| Treatment | level (mg/dL) |
| Vehicle | 56.8 ± 4.0 |
| Compound X (30 mg/kg) | 43.5 ± 4.1 |
| Combination of simvastatin (30 | 34.6 ± 2.1 |
| mg/kg) + ezetimibe (0.15 mg/kg) | |
| Triple combination of | 29.4 ± 2.5 |
| simvastatin (30 mg/kg) + | |
| ezetimibe (0.15 mg/kg) + | |
| Compound X (30 mg/kg) | |

Average ± standard error (N = 6)

### Conclusion:

By use in triple combination of Compound X, simvastatin and ezetimibe, Compound X showed an additional action of lowering the total cholesterol in plasma was observed (P<0.05, two-way ANOVA method).

As is obvious from the above results, a more effective prevention and/or treatment of hyperlipidemia can be carried out by a three-drug combined use of SSI, HMG-CoA reductase inhibitor and Ezetimibe.

In addition, when the combined use of three drugs is carried out, specific administration forms such as administration method and administration dosage form can be referred to the above-mentioned embodiment of the two-drug combined use of SSI and HMG-CoA reductase inhibitor.

### INDUSTRIAL APPLICABILITY

By the combined use of SSI and HMG-CoA reductase inhibitor in the present invention, hyperlipidemia of a mammal can be effectively prevented and/or treated.

1. A method for preventing and/or treating hyperlipidemia, which comprises administering to a mammal affected with hyperlipidemia a combination of an effective amount of squalene synthase inhibitor and HMG-CoA reductase inhibitor.

2. The method according to claim 1, wherein the HMG-CoA reductase inhibitor is administered at a high dose in approved dosage.

3. The method according to claim 2, wherein the HMG-CoA reductase inhibitor is administered at a maximum dose in approved dosage.

4. A method for preventing and/or treating hepatic toxicity caused by administration of HMG-CoA reductase inhibitor, which comprises administering an effective amount of squalene synthase inhibitor to inhibit toxicity caused by the administration of HMG-CoA reductase inhibitor to a mammal administered HMG-CoA reductase inhibitor.

5. The method according to claim 4, wherein the mammal is affected with hyperlipidemia.

6. The method according to claim 1 or 2, wherein the squalene synthase inhibitor is a compound represented by formula: 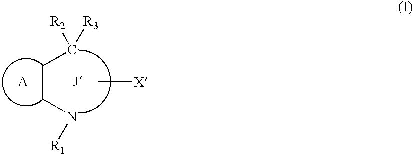
wherein, R₁ is a hydrogen atom or an optionally substituted hydrocarbon group, R₂ and R₃ are the same or different and a hydrogen atom, optionally substituted hydrocarbon group or an optionally substituted heterocyclic group, X′ is a group comprising an optionally esterified carboxyl group, an optionally substituted carbamoyl group, an optionally substituted hydroxy group, an optionally substituted amino group or an optionally substituted heterocyclic residue having a hydrogen atom which can be deprotonated, Ring A is an optionally substituted benzene ring or an optionally substituted heterocyclic ring, Ring J′ is a 7- or 8-membered heterocyclic ring containing 3 or less hetero atoms as ring constituent atoms, and Ring J′ may further have a substituent in addition to R₁, R₂, R₃ and X′.

7. The method according to claim 1 or 2, wherein the squalene synthase inhibitor is N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid.

8. The method according to claim 1 or 2, wherein the HMG-CoA reductase inhibitor is one or more drugs selected from the group consisting of atorvastatin, lovastatin, simvastatin, pravastatin, fluvastatin, rosuvastatin, cerivastatin and pitavastatin.

9. The method according to claim 1 or 2, wherein the mammal affected with hyperlipidemia is HMG-CoA reductase inhibitor intolerant patient.

10. The method according to claim 1 or 2, wherein the mammal affected with hyperlipidemia is a high-risk patient of ischemic heart disease.

11. The method according to claim 1 or 2, wherein the mammal affected with hyperlipidemia is a patient affected with familial hypercholesterolemia.

12. A pharmaceutical composition for a prevention and/or treatment of hyperlipidemia, which comprises combining an effective amount of squalene synthase inhibitor and HMG-CoA reductase inhibitor.

13. A pharmaceutical composition for a prevention and/or treatment of hyperlipidemia comprising an effective amount of squalene synthase inhibitor and HMG-CoA reductase inhibitor, which is compounded or packed so as to administer in divided doses, sequentially or simultaneously to a mammal affected with hyperlipidemia.

14. The pharmaceutical composition according to claim 12 or 13, which comprises combining with a high-dose in approved dosage of HMG-CoA reductase inhibitor.

15. The pharmaceutical composition according to claim 12 or 13, which comprises combining with a maximum dose in approved dosage of the HMG-CoA reductase inhibitor.

16. A method for enhancing an effect on prevention and/or treatment of hyperlipidemia by a HMG-CoA reductase inhibitor, which comprises administering an effective amount of squalene synthase inhibitor to a mammal affected with hyperlipidemia wherein an effective amount of HMG-CoA reductase inhibitor is administered.

17. Use of squalene synthase inhibitor for the manufacture of a pharmaceutical composition for preventing and/or treating hyperlipidemia which comprises combining an effective amount of squalene synthase inhibitor and HMG-CoA reductase inhibitor.

18. Use of squalene synthase inhibitor for the manufacture of a pharmaceutical composition for preventing and/or treating hyperlipidemia comprising an effective amount of squalene synthase inhibitor and HMG-CoA reductase inhibitor which is compounded or packed so as to administer in divided doses, sequentially or simultaneously to a mammal affected with hyperlipidemia.

19. The method according to claim 1 or 2, wherein an effective amount of ezetimibe is further administered in combination as third medicament.

20. The pharmaceutical composition according to claim 12 or 13, which comprises further combining an effective amount of ezetimibe as third medicament.

21. The use of squalene synthase inhibitor for the manufacture of a pharmaceutical composition for preventing and/or treating hyperlipidemia according to claim 17 or 18, which comprises further combining an effective amount of ezetimibe as third medicament.
